SEQUENCE LISTING (79 sequences)

SEQ ID NO: 1 - protein, 901 aa, Streptococcus agalactiae 2603
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPMTGGKGILSFILIGGAMMSIAGGIYIWKRYKKSSDMSIKKD

SEQ ID NO: 2 - protein, 194 aa, Streptococcus agalactiae 2603
NTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGK

SEQ ID NO: 3 - protein, 398 aa, Streptococcus agalactiae 2603
TIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNG

SEQ ID NO: 4 - protein, 251 aa, Streptococcus agalactiae 2603
QTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPMTGGKG

SEQ ID NO: 5 - protein, 896 aa, Streptococcus agalactiae H36B
MRKYQKFSKILTLSLFCLSQIPLNTNVLGESTVPENGAKGKLVVKKTDDQNKPLSKATFVLKPTSHSESKVEKVTTEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGKSIIKTINKDEPLDVVFVLDNSNSMKNNGKNNKAKKAGEAVETIIKDVLGANVENRAALVTYGSDIFDGRTVKVIKGFKEDPYYGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVKGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSDGEITELMNSFSSKPEYYTPIVTSADVSNNEILSKIQQQFEKILTKENSIVNGTIEDPMGDKINLHLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILSFILIGGAMMSIAGGIYIWKRHKKSSDASIEKD

SEQ ID NO: 6 - protein, 194 aa, Streptococcus agalactiae H36B
NTNVLGESTVPENGAKGKLVVKKTDDQNKPLSKATFVLKPTSHSESKVEKVTTEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGK

SEQ ID NO: 7 - protein, 393 aa, Streptococcus agalactiae H36B
SIIKTINKDEPLDVVFVLDNSNSMKNNGKNNKAKKAGEAVETIIKDVLGANVENRAALVTYGSDIFDGRTVKVIKGFKEDPYYGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVKGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSDGEITELMNSFSSKPEYYTPIVTSADVSNNEILSKIQQQFEKILTKENSIVNGTIEDPMGDKINLHLGNG

SEQ ID NO: 8 - protein, 251 aa, Streptococcus agalactiae H36B
QTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKG

SEQ ID NO: 9 - protein, 901 aa, Streptococcus agalactiae CJB111
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTFYRNGPVREHGTPTKLYINSLKQKNYDIFNFGIDISGFRQVYNEDYKKNQDGTFQKLKEEAFELSDGEITELMKSFSSKPEYYTPIVTSSDASNNEILSKIQQQFEKILTKENSIVNGTIEDPMGDKINLQLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEDPNTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILSFILIGGSMMSIAGGIYIWKRYKKSSDISREKD

SEQ ID NO: 10 - protein, 194 aa, Streptococcus agalactiae CJB111; identical to SEQ ID NO: 2

SEQ ID NO: 11 - protein, 398 aa, Streptococcus agalactiae CJB111
TIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTFYRNGPVREHGTPTKLYINSLKQKNYDIFNFGIDISGFRQVYNEDYKKNQDGTFQKLKEEAFELSDGEITELMKSFSSKPEYYTPIVTSSDASNNEILSKIQQQFEKILTKENSIVNGTIEDPMGDKINLQLGNG

SEQ ID NO: 12 - protein, 251 aa, Streptococcus agalactiae CJB111
QTLQPSDYTLQGNDGSIMKDSIATGGPNNDGGILKGVKLEYIKNKLYVRGLNLGEGQKVTLTYDVKLDDSFISNKFYDTNGRTTLNPKSEDPNTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKG

SEQ ID NO: 13 - protein, 901 aa, Streptococcus agalactiae 515
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPDDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQILQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPKTGGKGILSFILIGGAMMSIAGGIYIWKRYKKSSDMSIKKD

SEQ ID NO: 14 - protein, 194 aa, Streptococcus agalactiae 515; identical to SEQ ID NO: 2

SEQ ID NO: 15 - protein, 398 aa, Streptococcus agalactiae 515
TIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPDDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNG

SEQ ID NO: 16 - protein, 251 aa, Streptococcus agalactiae 515
QILQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPKTGGKG

SEQ ID NO: 17 - protein, 901 aa, Streptococcus agalactiae NEM316
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNHEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPRAKWGSTTNGLTPEQQKQYYLSKVGETFTMKAFMEADDILSQVDRNSQKIIVHITDGVPTRSYAINNFKLGASYESQFEQMKKNGYLNKSNFLLTDKPEDIKGNGESYFLFPLDSYQTQIISGNLQKLHYLDLNLNYPKGTIYRNGPVREHGTPTKLYINSLKQKNYDIFNFGIDISAFRQVYNEDYKKNQDGTFQKLKEEAFELSDGEITELMKSFSSKPEYYTPIVTSSDASNNEILSKIQQQFEKVLTKENSIVNGTIEDPMGDKINLQLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEDPNTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILSFILIGGSMMSIAGGIYIWKRYKKSSDISREKD

SEQ ID NO: 18 - protein, 194 aa, Streptococcus agalactiae NEM316
NTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNHEELDKQYPPTGIYEDTKESYKLEHVKGSVPNGKSEAKAVNPYSSEGEHIREIPEGTLSKRISEVGDLAHNKYKIELTVSGK

SEQ ID NO: 19 - protein, 398 aa, Streptococcus agalactiae NEM316
TIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPRAKWGSTTNGLTPEQQKQYYLSKVGETFTMKAFMEADDILSQVDRNSQKIIVHITDGVPTRSYAINNFKLGASYESQFEQMKKNGYLNKSNFLLTDKPEDIKGNGESYFLFPLDSYQTQIISGNLQKLHYLDLNLNYPKGTIYRNGPVREHGTPTKLYINSLKQKNYDIFNFGIDISAFRQVYNEDYKKNQDGTFQKLKEEAFELSDGEITELMKSFSSKPEYYTPIVTSSDASNNEILSKIQQQFEKVLTKENSIVNGTIEDPMGDKINLQLGNG

SEQ ID NO: 20 - protein, 251 aa, Streptococcus agalactiae NEM316; identical to SEQ ID NO: 12

SEQ ID NO: 21 - protein, 896 aa, Streptococcus agalactiae DK21
MRKYQKFSKILTLSLFCLSQIPLNTNVLGESTVPENGAKGKLVVKKTDDQNKPLSKATFVLKPTSHSESKVEKVTTEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGKSIIKTINKDEPLDVVFVLDNSNSMKNNGKNNKAKKAGEAVETIIKDVLGANVENRAALVTYGSDIFDGRTVKVIKGFKEDPYYGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVTGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSDGEITELMNSFSSKPEYYTPIVTSADVSNNEILSKIQQQFEKILTKENSIVNGTIEDPMGDKINLQLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILSFILIGGAMMSIAGGIYIWKRHKKSSDASIEKD

SEQ ID NO: 22 - protein, 194 aa, Streptococcus agalactiae DK21; identical to SEQ ID NO: 6

SEQ ID NO: 23 - protein, 393 aa, Streptococcus agalactiae DK21
SIIKTINKDEPLDVVFVLDNSNSMKNNGKNNKAKKAGEAVETIIKDVLGANVENRAALVTYGSDIFDGRTVKVIKGFKEDPYYGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVTGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSDGEITELMNSFSSKPEYYTPIVTSADVSNNEILSKIQQQFEKILTKENSIVNGTIEDPMGDKINLQLGNG

SEQ ID NO: 24 - protein, 251 aa, Streptococcus agalactiae DK21; identical to SEQ ID NO: 8

SEQ ID NO: 25 - protein, 896 aa, Streptococcus agalactiae CJB110
MRKYQKFSKILTLSLFCLSQIPLNTNVLGESTVPENGAKGKLVVKKTDDQNKPLSKATFVLKTTAHPESKIEKVTAEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGKSIIKTINKDEPLDVVFVLDNSNSMKNNGKNNKAKKAGEAVETIIKDVLGANVENRAALVTYGSDIFDGRTVKVIKGFKEDPYHGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVTGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSGGEITELMKSFSSKPEYYTPIVTSADVSNNEILSKIQQQFEKILTKENSIVNGTIEDPMGDKINLQLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILSFILIGGAMMSIAGGIYIWKKHKKSSDASIEKD

SEQ ID NO: 26 - protein, 194 aa, Streptococcus agalactiae CJB110
NTNVLGESTVPENGAKGKLVVKKTDDQNKPLSKATFVLKTTAHPESKIEKVTAEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGK

SEQ ID NO: 27 - protein, 393 aa, Streptococcus agalactiae CJB110
SIIKTINKDEPLDVVFVLDNSNSMKNNGKNNKAKKAGEAVETIIKDVLGANVENRAALVTYGSDIFDGRTVKVIKGFKEDPYHGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVTGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSGGEITELMKSFSSKPEYYTPIVTSADVSNNEILSKIQQQFEKILTKENSIVNGTIEDPMGDKINLQLGNG

SEQ ID NO: 28 - protein, 251 aa, Streptococcus agalactiae CJB110; identical to SEQ ID NO: 8

SEQ ID NO: 29 - protein, 4 aa, Artificial Sequence
  Linker
GSGS

SEQ ID NO: 30 - protein, 6 aa, Artificial Sequence
  Linker
GSGGGG

SEQ ID NO: 31 - protein, 8 aa, Artificial Sequence
  Linker
GSGSGGGG

SEQ ID NO: 32 - DNA, 26 bp, Artificial Sequence
  IC adjuvant
ncncncncncncncncncncncncnc

SEQ ID NO: 33 - protein, 11 aa, Artificial Sequence
  Polycationic peptide adjuvant
KLKLLLLLKLK

SEQ ID NO: 34 - protein, 554 aa, Streptococcus agalactiae 2603
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 35 - protein, 517 aa, Streptococcus agalactiae 2603
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 36 - protein, 525 aa, Streptococcus agalactiae 2603
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTG

SEQ ID NO: 37 - protein, 520 aa, Streptococcus agalactiae 2603
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 38 - protein, 483 aa, Streptococcus agalactiae 2603
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 39 - protein, 271 aa, Streptococcus agalactiae 2603
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKG

SEQ ID NO: 40 - protein, 705 aa, Streptococcus agalactiae 2603
MKRINKYFAMFSALLLTLTSLLSVAPAFADEATTNTVTLHKILQTESNLNKSNFPGTTGLNGKDYKGGAISDLAGYFGEGSKEIEGAFFALALKEDKSGKVQYVKAKEGNKLTPALINKDGTPEITVNIDEAVSGLTPEGDTGLVFNTKGLKGEFKIVEVKSKSTYNNNGSLLAASKAVPVNITLPLVNEDGVVADAHVYPKNTEEKPEIDKNFAKTNDLTALTDVNRLLTAGANYGNYARDKATATAEIGKVVPYEVKTKIHKGSKYENLVWTDIMSNGLTMGSTVSLKASGTTETFAKDTDYELSIDARGFTLKFTADGLGKLEKAAKTADIEFTLTYSATVNGQAIIDNPESNDIKLSYGNKPGKDLTELPVTPSKGEVTVAKTWSDGIAPDGVNVVYTLKDKDKTVASVSLTKTSKGTIDLGNGIKFEVSGNFSGKFTGLENKSYMISERVSGYGSAINLENGKVTITNTKDSDNPTPLNPTEPKVETHGKKFVKTNEQGDRLAGAQFVVKNSAGKYLALKADQSEGQKTLAAKKIALDEAIAAYNKLSATDQKGEKGITAKELIKTKQADYDAAFIEARTAYEWITDKARAITYTSNDQGQFEVTGLADGTYNLEETLAPAGFAKLAGNIKFVVNQGSYITGGNIDYVANSNQKDATRVENKKVTIPQTGGIGTILFTIIGLSIMLGAVVIMKRRQSKEA

SEQ ID NO: 41 - protein, 675 aa, Streptococcus agalactiae 515
MKKINKYFAVFSALLLTVTSLFSVAPVFAEEAKTTDTVTLHKIVMPRTAFDGFTAGTKGKDNTDYVGKQIEDLKTYFGSGEAKEIAGAYFAFKNEAGTKYITENGEEVDTLDTTDAKGCAVLKGLTTDNGFKFNTSKLTGTYQIVELKEKSTYNNDGSILADSKAVPVKITLPLVNDNGVVKDAHVYPKNTETKPQVDKNFADKELDYANNKKDKGTVSASVGDVKKYHVGTKILKGSDYKKLIWTDSMTKGLTFNNDIAVTLDGATLDATNYKLVADDQGFRLVLTDKGLEAVAKAAKTKDVEIKITYSATLNGSAVVEVLETNDVKLDYGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYEWVEDKNAKNVVKLISNDKGQFEITGLTEGQYSLEETQAPTGYAKLSGDVSFNVNATSYSKGSAQDIEYTQGSKTKDAQQVINKKVTIPQTGGIGTIFFTIIGLSIMLGAVVIMKRRQSEEV

SEQ ID NO: 42 - protein, 674 aa, Streptococcus agalactiae CJB111
MKKINKCLTMFSTLLLILTSLFSVAPAFADDATTDTVTLHKIVMPQAAFDNFTEGTKGKNDSDYVGKQINDLKSYFGSTDAKEIKGAFFVFKNETGTKFITENGKEVDTLEAKDAEGGAVLSGLTKDNGFVFNTAKLKGIYQIVELKEKSNYDNNGSILADSKAVPVKITLPLVNNQGVVKDAHIYPKNTETKPQVDKNFADKDLDYTDNRKDKGVVSATVGDKKEYIVGTKILKGSDYKKLVWTDSMTKGLTFNNNVKVTLDGEDFPVLNYKLVTDDQGFRLALNATGLAAVAAAAKDKDVEIKITYSATVNGSTTVEIPETNDVKLDYGNNPTEESEPQEGTPANQEIKVIKDWAVDGTITDANVAVKAIFTLQEKQTDGTWVNVASHEATKPSRFEHTFTGLDNAKTYRVVERVSGYTPEYVSFKNGVVTIKNNKNSNDPTPINPSEPKVVTYGRKFVKTNQANTERLAGATFLVKKEGKYLARKAGAATAEAKAAVKTAKLALDEAVKAYNDLTKEKQEGQEGKTALATVDQKQKAYNDAFVKANYSYEWVADKKADNVVKLISNAGGQFEITGLDKGTYGLEETQAPAGYATLSGDVNFEVTATSYSKGATTDIAYDKGSVKKDAQQVQNKKVTIPQTGGIGTILFTIIGLSIMLGAVVIMKKRQSEEA

SEQ ID NO: 43 - protein, 693 aa, Streptococcus agalactiae H36B
MKRINKYFAMFSALLLILTSLLSVAPVFAAEMGNITKTVTLHKIVQTSDNLAKPNFPGINGLNGTKYMGQKLTDISGYFGQGSKEIAGAFFAVMNESQTKYITESGTEVESIDAAGVLKGLTTENGITFNTANLKGTYQIVELLDKSNYKNGDKVLADSKAVPVKITLPLYNEEGIVVDAEVYPKNTEEAPQIDKNFAKANKLLNDSDNSAIAGGADYDKYQAEKAKATAEIGQEIPYEVKTKIQKGSKYKNLAWVDTMSNGLTMGNTVNLEASSGSFVEGTDYNVERDDRGFTLKFTDTGLTKLQKEAETQAVEFTLTYSATVNGAAIDDKPESNDIKLQYGNKPGKKVKEIPVTPSNGEITVSKTWDKGSDLENANVVYTLKDGGTAVASVSLTKTTPNGEINLGNGIKFTVTGAFAGKFSGLTDSKTYMISERIAGYGNTITTGAGSAAITNTPDSDNPTPLNPTEPKVVTHGKKFVKTSSTETERLQGAQFVVKDSAGKYLALKSSATISAQTTAYTNAKTALDAKIAAYNKLSADDQKGTKGETAKAEIKTAQDAYNAAFIVARTAYEWVTNKEDANVVKVTSNADGQFEVSGLATGDYKLEETQAPAGYAKLAGDVDFKVGNSSKADDSGNIDYTASSNKKDAQRIENKKVTIPQTGGIGTILFTIIGLSIMLGAVIIMKRRQSEEA

SEQ ID NO: 44 - protein, 704 aa, Streptococcus agalactiae CJB110
MKKINKYFAVFSALLLTVTSLLSVAPAFADEATTNTVTLHKILQTESNLNKSNFPGTTGLNGDDYKGESISDLAEYFGSGSKEIDGAFFALALEEEKDGVVQYVKAKANDKLTPDLITKGTPATTTKVEEAVGGLTTGTGIVFNTAGLKGNFKIIELKDKSTYNNNGSLLAASKAVPVKITLPLVSKDGVVKDAHVYPKNTETKPEVDKNFAKTNDLTALKDATLLKAGADYKNYSATKATVTAEIGKVIPYEVKTKVLKGSKYEKLVWTDTMSNGLTMGDDVNLAVSGTTTTFIKDIDYTLSIDDRGFTLKFKATGLDKLEEAAKASDVEFTLTYKATVNGQAIIDNPEVNDIKLDYGNKPGTDLSEQPVTPEDGEVKVTKTWAAGANKADAKVVYTLKNATKQVVASVALTAADTKGTINLGKGMTFEITGAFSGTFKGLQNKAYTVSERVAGYTNAINVTGNAVAITNTPDSDNPTPLNPTQPKVETHGKKFVKVGDADARLAGAQFVVKNSAGKFLALKEDAAVSGAQTELATAKTDLDNAIKAYNGLTKAQQEGADGTSAKELINTKQSAYDAAFIKARTAYTWVDEKTKAITFTSNNQGQFEVTGLEVGSYKLEETLAPAGYAKLSGDIEFTVGHDSYTSGDIKYKTDDASNNAQKVFNKKVTIPQTGGIGTILFTIIGLSIMLGAVVIMKRRQSEEA

SEQ ID NO: 45 - protein, 682 aa, Streptococcus agalactiae DK21
MKKINKFFVAFSALLLILTSLLSVAPAFAEEERTTETVTLHKILQTETNLKNSAFPGTKGLDGTEYDGKAIDKLDSYFGNDSKDIGGAYFILANSKGEYIKANDKNKLKPEFSGNTPKTTLNISEAVGGLTEENAGIKFETTGLRGDFQIIELKDKSTYNNGGAILADSKAVPVKITLPLINKDGVVKDAHVYPKNTETKPQIDKNFADKNLDYINNQKDKGTISATVGDVKKYTVGTKILKGSDYKKLVWTDSMTKGLTFNNDVTVTLDGANFEQSNYTLVADDQGFRLVLNATGLSKVAEAAKTKDVEIKINYSATVNGSTVVEKSENNDVKLDYGNNPTTENEPQTGNPVNKEITVRKTWAVDGNEVNKGDEKVDAVFTLQVKDSDKWVNVDSATATAATDFKYTFKNLDNAKTYRVVERVSGYAPAYVSFVGGVVTIKNNKNSNDPTPINPSEPKVVTYGRKFVKTNQDGSERLAGATFLVKNSQSQYLARKSGVATNEAHKAVTDAKVQLDEAVKAYNKLTKEQQESQDGKAALNLIDEKQTAYNEAFAKANYSYEWVVDKNAANVVKLISNTAGKFEITGLNAGEYSLEETQAPTGYAKLSSDVSFKVNDTSYSEGASNDIAYDKDSGKTDAQKVVNKKVTIPQTGGIGTILFTIIGLSIMLGAVVIMKRRQSEEA

SEQ ID NO: 46 - protein, 502 aa, Streptococcus agalactiae COH1
MKKKMIQSLLVASLAFGMAVSPVTPIAFAAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTELPSTGGIGTTIFYIIGAILVIGAGIVLVARRRLRS

SEQ ID NO: 47 - protein, 438 aa, Streptococcus agalactiae COH1
AETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTE

SEQ ID NO: 48 - protein, 438 aa, Streptococcus agalactiae COH1
AETGTITVQDTKKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTE

SEQ ID NO: 49 - protein, 941 aa, Artificial Sequence
  GBS80-GBS1523 hybrid
MASAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTEGGGGSGGGGSGGGGSELAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 50 - protein, 932 aa, Artificial Sequence
  GBS80-GBS1523 hybrid
MASAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTEGSGGGGELAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 51 - protein, 941 aa, Artificial Sequence
  GBS80-GBS1523 hybrid
MASAETGTITVQDTKKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTEGGGGSGGGGSGGGGSELAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 52 - protein, 931 aa, Artificial Sequence
  GBS80-GBS1523 hybrid
MASAETGTITVQDTKKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTEGSGGGGELAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 53 - protein, 890 aa, Streptococcus agalactiae 2603
MKKRQKIWRGLSVTLLILSQIPFGILVQGETQDTNQALGKVIVKKTGDNATPLGKATFVLKNDNDKSETSHETVEGSGEATFENIKPGDYTLREETAPIGYKKTDKTWKVKVADNGATIIEGMDADKAEKRKEVLNAQYPKSAIYEDTKENYPLVNVEGSKVGEQYKALNPINGKDGRREIAEGWLSKKITGVNDLDKNKYKIELTVEGKTTVETKELNQPLDVVVLLDNSNSMNNERANNSQRALKAGEAVEKLIDKITSNKDNRVALVTYASTIFDGTEATVSKGVADQNGKALNDSVSWDYHKTTFTATTHNYSYLNLTNDANEVNILKSRIPKEAEHINGDRTLYQFGATFTQKALMKANEILETQSSNARKKLIFHVTDGVPTMSYAINFNPYISTSYQNQFNSFLNKIPDRSGILQEDFIINGDDYQIVKGDGESFKLFSDRKVPVTGGTTQAAYRVPQNQLSVMSNEGYAINSGYIYLYWRDYNWVYPFDPKTKKVSATKQIKTHGEPTTLYFNGNIRPKGYDIFTVGIGVNGDPGATPLEAEKFMQSISSKTENYTNVDDTNKIYDELNKYFKTIVEEKHSIVDGNVTDPMGEMIEFQLKNGQSFTHDDYVLVGNDGSQLKNGVALGGPNSDGGILKDVTVTYDKTSQTIKINHLNLGSGQKVVLTYDVRLKDNYISNKFYNTNNRTTLSPKSEKEPNTIRDFPIPKIRDVREFPVLTISNQKKMGEVEFIKVNKDKHSESLLGAKFQLQIEKDFSGYKQFVPEGSDVTTKNDGKIYFKALQDGNYKLYEISSPDGYIEVKTKPVVTFTIQNGEVTNLKADPNANKNQIGYLEGNGKHLITNTPKRPPGVFPKTGGIGTIVYILVGSTFMILTICSFRRKQL

SEQ ID NO: 54 - protein, 1434 aa, Streptococcus agalactiae COH1
MLKKCQTFIIESLKKKKHPKEWKIIMWSLMILTTFLTTYFLILPAITVEETKTDDVGITLENKNSSQVTSSTSSSQSSVEQSKPQTPASSVTETSSSEEAAYREEPLMFRGADYTVTVTLTKEAKIPKNADLKVTELKDNSATFKDYKKKALTEVAKQDSEIKNFKLYDITIESNGKEAEPQAPVKVEVNYDKPLEASDENLKVVHFKDDGQTEVLKSKDTAETKNTSSDVAFKTDSFSIYAIVQEDNTEVPRLTYHFQNNDGTDYDFLTASGMQVHHQIIKDGESLGEVGIPTIKAGEHFNGWYTYDPTTGKYGDPVKFGEPITVTETKEICVRPFMSKVATVTLYDDSAGKSILERYQVPLDSSGNGTADLSSFKVSPPTSTLLFVGWSKTQNGAPLSESEIQALPVSSDISLYPVFKESYGVEFNTGDLSTGVTYIAPRRVLTGQPASTIKPNDPTRPGYTFAGWYTAASGGAAFDFNQVLTKDTTLYAHWSPAQTTYTINYWQQSATDNKNATDAQKTYEYAGQVTRSGLSLSNQTLTQQDINDKLPTGFKVNNTRTETSVMIKDDGSSVVNVYYDRKLITIKFAKYGGYSLPEYYYSYNWSSDADTYTGLYGTTLAANGYQWKTGAWGYLANVGNNQVGTYGMSYLGEFILPNDTVDSDVIKLFPKGNIVQTYRFFKQGLDGTYSLADTGGGAGADEFTFTEKYLGFNVKYYQRLYPDNYLFDQYASQTSAGVKVPISDEYYDRYGAYHKDYLNLVVWYERNSYKIKYLDPLDNTELPNFPVKDVLYEQNLSSYAPDTTTVQPKPSRPGYVWDGKWYKDQAQTQVFDFNTTMPPHDVKVYAGWQKVTYRVNIDPNGGRLSKTDDTYLDLHYGDRIPDYTDITRDYIQDPSGTYYYKYDSRDKDPDSTKDAYYTTDTSLSNVDTTTKYKYVKDAYKLVGWYYVNPDGSIRPYNFSGAVTQDINLRAIWRKAGDYHIIYSNDAVGTDGKPALDASGQQLQTSNEPTDPDSYDDGSHSALLRRPTMPDGYRFRGWWYNGKIYNPYDSIDIDAHLADANKNITIKPVIIPVGDIKLEDTSIKYNGNGGTRVENGNVVTQVETPRMELNSTTTIPENQYFTRTGYNLIGWHHDKDLADTGRVEFTAGQSIGIDNNPDATNTLYAVWQPKEYTVRVSKTVVGLDEDKTKDFLFNPSETLQQENFPLRDGQTKEFKVPYGTSISIDEQAYDEFKVSESITEKNLATGEADKTYDATGLQSLTVSGDVDISFTNTRIKQKVRLQKVNVENDNNFLAGAVFDIYESDANGNKASHPMYSGLVTNDKGLLLVDANNYLSLPVGKYYLTETKAPPGYLLPKNDISVLVISTGVTFEQNGNNATPIKENLVDGSTVYTFKITNSKGTELPSTGGIGTHIYILVGLALALPSGLILYYRKKI

SEQ ID NO: 55 - protein, 478 aa, Streptococcus agalactiae 2603
MNNNEKKVKYFLRKTAYGLASMSAAFAVCSGIVHADTSSGISASIPHKKQVNLGAVTLKNLISKYRGNDKAIAILLSRVNDFNRASQDTLPQLINSTEAEIRNILYQGQIGKQNKPSVTTHAKVSDQELGKQSRRSQDIIKSLGFLSSDQKDILVKSISSSKDSQLILKFVTQATQLNNAESTKAKQMAQNDVALIKNISPEVLEEYKEKIQRASTKSQVDEFVAEAKKVVNSNKETLVNQANGKKQEIAKLENLSNDEMLRYNTAIDNVVKQYNEGKLNITAAMNALNSIKQAAQEVAQKNLQKQYAKKIERISSKGLALSKKAKEIYEKHKSILPTPGYYADSVGTYLNRFRDKQTFGNRSVWTGQSGLDEAKKMLDEVKKLLKELQDLTRGTKEDKKPDVKPEAKPEAKPDVKPEAKPDVKPEAKPDVKPEAKPDVKPEAKPDVKPEAKPDVKIVSLIVMLSAGLITIVLKHKKN

SEQ ID NO: 56 - protein, 370 aa, Streptococcus agalactiae 2603
TSSGISASIPHKKQVNLGAVTLKNLISKYRGNDKAIAILLSRVNDFNRASQDTLPQLINSTEAEIRNILYQGQIGKQNKPSVTTHAKVSDQELGKQSRRSQDIIKSLGFLSSDQKDILVKSISSSKDSQLILKFVTQATQLNNAESTKAKQMAQNDVALIKNISPEVLEEYKEKIQRASTKSQVDEFVAEAKKVVNSNKETLVNQANGKKQEIAKLENLSNDEMLRYNTAIDNVVKQYNEGKLNITAAMNALNSIKQAAQEVAQKNLQKQYAKKIERISSKGLALSKKAKEIYEKHKSILPTPGYYADSVGTYLNRFRDKQTFGNRSVWTGQSGLDEAKKMLDEVKKLLKELQDLTRGTKEDKKPDVKPE

SEQ ID NO: 57 - protein, 411 aa, Streptococcus agalactiae 2603
TSSGISASIPHKKQVNLGAVTLKNLISKYRGNDKAIAILLSRVNDFNRASQDTLPQLINSTEAEIRNILYQGQIGKQNKPSVTTHAKVSDQELGKQSRRSQDIIKSLGFLSSDQKDILVKSISSSKDSQLILKFVTQATQLNNAESTKAKQMAQNDVALIKNISPEVLEEYKEKIQRASTKSQVDEFVAEAKKVVNSNKETLVNQANGKKQEIAKLENLSNDEMLRYNTAIDNVVKQYNEGKLNITAAMNALNSIKQAAQEVAQKNLQKQYAKKIERISSKGLALSKKAKEIYEKHKSILPTPGYYADSVGTYLNRFRDKQTFGNRSVWTGQSGLDEAKKMLDEVKKLLKELQDLTRGTKEDKKPDVKPEAKPEAKLEAKPEAKPATKKSVNTSGNLAAKKAIENKKYSKK

SEQ ID NO: 58 - protein, 436 aa, Streptococcus agalactiae 2603
MNNNEKKVKYFLRKTAYGLASMSAAFAVCSGIVHADTSSGISASIPHKKQVNLGAVTLKNLISKYRGNDKAIAILLSRVNDFNRASQDTLPQLINSTEAEIRNILYQGQIGKQNKPSVTTHAKVSDQELGKQSRRSQDIIKSLGFLSSDQKDILVKSISSSKDSQLILKFVTQATQLNNAESTKAKQMAQNDVALIKNISPEVLEEYKEKIQRASTKSQVDEFVAEAKKVVNSNKETLVNQANGKKQEIAKLENLSNDEMLRYNTAIDNVVKQYNEGKLNITAAMNALNSIKQAAQEVAQKNLQKQYAKKIERISSKGLALSKKAKEIYEKHKSILPTPGYYADSVGTYLNRFRDKQTFGNRSVWTGQSGLDEAKKMLDEVKKLLKELQDLTRGTKEDKKPDVKPEAKPEAKPDVKPEAKPDVKPEAKPDVKPEAK

SEQ ID NO: 59 - protein, 364 aa, Streptococcus agalactiae 2603
TSSGISASIPHKKQVNLGAVTLKNLISKYRGNDKAIAILLSRVNDFNRASQDTLPQLINSTEAEIRNILYQGQIGKQNKPSVTTHAKVSDQELGKQSRRSQDIIKSLGFLSSDQKDILVKSISSSKDSQLILKFVTQATQLNNAESTKAKQMAQNDVALIKNISPEVLEEYKEKIQRASTKSQVDEFVAEAKKVVNSNKETLVNQANGKKQEIAKLENLSNDEMLRYNTAIDNVVKQYNEGKLNITAAMNALNSIKQAAQEVAQKNLQKQYAKKIERISSKGLALSKKAKEIYEKHKSILPTPGYYADSVGTYLNRFRDKQTFGNRSVWTGQSGLDEAKKMLDEVKKLLKELQDLTRGTKEDKK

SEQ ID NO: 60 - protein, 518 aa, Streptococcus agalactiae 2603
MNNNEKKVKYFLRKTAYGLASMSAAFAVCSGIVHADTSSGISASIPHKKQVNLGAVTLKNLISKYRGNDKAIAILLSRVNDFNRASQDTLPQLINSTEAEIRNILYQGQIGKQNKPSVTTHAKVSDQELGKQSRRSQDIIKSLGFLSSDQKDILVKSISSSKDSQLILKFVTQATQLNNAESTKAKQMAQNDVALIKNISPEVLEEYKEKIQRASTKSQVDEFVAEAKKVVNSNKETLVNQANGKKQEIAKLENLSNDEMLRYNTAIDNVVKQYNEGKLNITAAMNALNSIKQAAQEVAQKNLQKQYAKKIERISSKGLALSKKAKEIYEKHKSILPTPGYYADSVGTYLNRFRDKQTFGNRSVWTGQSGLDEAKKMLDEVKKLLKELQDLTRGTKEDKKPDVKPEAKPEAKPDVKPEAKPDVKPEAKPDVKPEAKPDVKPEAKPDVKPEAKPDVKEAKPDVKPEAKPEAKPEAKSEAKPEAKLEAKPEAKPATKKSVNTSGNLAAKKAIENKKYSKK

SEQ ID NO: 61 - protein, 378 aa, Streptococcus agalactiae 515
MNNNEKKVKYFLRKTAYGLASMSAAFAVCSGIVHADTSSGISASIPHKKQVNLGAVTLKNLISKYRGNDKAIAILLSRVNDFNRASQDTLPQLINSTEAEIRNILYQGQIGKQNKPSVTTHAKVSDQELGKQSRRSQDIIKSLGFLSSDQKDILVKSISSSKDSQLILKFVTQATQLNNAESTKAKQMAQNDVALIKNISPEVLEEYKEKIQRASTKSQVDEFVAEAKKVVNSNKETLVNQANGKKQEIAKLENLSNDEMLRYNTAIDNVVKQYNEGKLNITAAMNALNSIKQAAQEVAQKNLQKQYAKKIERISSKGLALSKKAKEIYEKHKSILPTPGYYADSVGTYLNRFRDKQTFGNRSVWTGQSGLDEAKKNA

SEQ ID NO: 62 - protein, 471 aa, Streptococcus agalactiae CJB111
MNNNEKKVKYFLRKTAYGLASMSAAFAVCSGIVHADTSSGISDSIPHKKQVNLGAVTLKNLISKYRGNDKAIAILLSRVDDFNRASQDTLPQLINSTEAEINNTLPQGRIIKQSIPVVRLKVERLGSGAIKAESINNIKAESINKIQGKSTNTIKAESINKIKVESINTIKAESINKIQAKPINTIKAESINTIKAESIHKIKPQSIKSTSATHVKVSDQELAKQSRRSQDIIKSLGFLSSDQKDILVKSISSSKDSQLILKFVTQATQLNNAESTKAKHMAQNDVASIKNISLEVLEEYKEKIQRASTKSQVDELVAEAKKVVNSNKETLVNQANGKKQEIAKLENLSNDEMLRYNTAIDNVVKQYNEGKLNITDAMNALNSIKQAAQEVAQKNLQKQYAKKIERISLKGLALSKKAKEIYEKHKSILPTPGYYADSVGTYLNRFRDKRTFGNRSVWTGQSGLDEAKKNA

SEQ ID NO: 63 - protein, 522 aa, Streptococcus agalactiae COH1
MNNNEKKVKYFLRKTAYGLASMSAAFIVCSGIVNTPTVSADSPDTLKVEKLGKLKDVKSVHELTPISIPNELKGAKEQALSSIISHPNITNSEVDKLASDYSFRINTSNDVNDVKRLLNEFYNAVARKQLDTNSADYRSKIDNISTTGLAIALEAKEIYEANKSILPHRYKDSVGTYVNSFEERRSPGKFNIWNGQEGFNAAQKLLEDVKKLLLELQNLTKNNKPNIQVPKQAPTEAAKPALSPEALTRLTTWYNQAKDLLKDDQVKDKYVDILSVQKAVDQAYDHVEEGKFITTDQANQLANKLRDALQSLELKDKKVAKPVAKGTYDVKYVDTEGKEVAKSRHFEGEEGAAFVTSAKEVAGYKLVRTEGAVSNVFTAGAQVRTYVYEKVKPEVKPDVKPEAKPEAKPEVKPDVKPEAKPEAKPEVKSDVKPEAKPEAKPEAKPEVKPDVKPEAKPEAKPATKKSVNTSGNLVAKKAIENKKYSKKLPSTGEAASPLLAIVSLIVMLSAGLITIVLKHKKN

SEQ ID NO: 64 - protein, 1085 aa, Streptococcus agalactiae COH1
MLKKQFGNFGEKSRKVRVKMRKSGKHWVKSVMTQIGYVILSRFSGKEKSSKVQTTSEDLSRTKTSASILTAVAALGAVVGGTTDTTSVSAEETPTATELTGNEKTLATAETVVVAPEVKTVNSDSSSHSTSESQSMSTSTLQSTSASLSASESLMDSTSASLSESSSLSEYSSLSLSSSESVSASESVQSSEAATTARVQPRAMRVVSSASDMETLPAALISGEGDVTTVQGQDVTDKLQNLDIKLSGGVQAKAGVINMDKSESMHMSLKFTIDSVNRGDTFEIKLSDNIDTNGASNYSIVEPIKSPTGEVYATGIYDSQKKSIVYSFTDFAASKNNINGILDIPLWPDDTTVQNTKEDVLFSVKIKDQEATIKETVKYDPPVRIDFAGGVSVDSRITNIDDVGKKMTYISQINVDGKSLYNYNGLYTRIYNYSKESTADLKNSTIKIYKTTSDNIVESMVQDYSSMEDVTSKFANSYPEKGWYDIYWGQFIASNETYVIVVETPFTNAVTLNTTLSDYNENNGVEHNHTYSSESGYSDVNAQERKILSELVSSSESVSSSESVSNSESISTSESVSNSESISSSESVSSSESISTSESVSTSESISSSESVSSSESVSSSESISSSESVSNSESISSSESVSNSESISSSESVSSSESISNSESISSSESVSTSESISSSESVSNSESISSSESVSSSESVSTSESISSSESVSNSESISTSESVSTSESISSSESVSSSESISSSESVSNSESISNSESVSSSESVSNSESISSSESVSNSESISTSESVSTSESISSSESVSNSESISSSESVSNSESISSSESVSNSESISSSESVSNSESISSSESVSSSESVSSSESISTSESVSNSESISSSESVSNSESISSSESVSNSESISSSESVSNSESISSSESVSSSESISSSESVSSSESVSNSESISSSESVSNSESISSSESVSSSESISSSESVSNSESILSSESVSSSESISSSESISSSESVSMSTTESLSESEVSGDSEISSSTESSSQSESMNHTEIKSDSESQHEVKHQVLPETGDNSASALGLLGAGLLLGATKSRKKKKD

SEQ ID NO: 65 - protein, 1233 aa, Streptococcus agalactiae 2603
MDKHHSKKAILKLTLITTSILLMHSNQVNAEEQELKNQEQSPVIANVAQQPSPSVTTNTVEKTSVTAASASNTAKEMGDTSVKNDKTEDELLEELSKNLDTSNLGADLEEEYPSKPETTNNKESNVVTNASTAIAQKVPSAYEEVKPESKSSLAVLDTSKITKLQAITQRGKGNVVAIIDTGFDINHDIFRLDSPKDDKHSFKTKTEFEELKAKHNITYGKWVNDKIVFAHNYANNTETVADIAAAMKDGYGSEAKNISHGTHVAGIFVGNSKRPAINGLLLEGAAPNAQVLLMRIPDKIDSDKFGEAYAKAITDAVNLGAKTINMSIGKTADSLIALNDKVKLALKLASEKGVAVVVAAGNEGAFGMDYSKPLSTNPDYGTVNSPAISEDTLSVASYESLKTISEVVETTIEGKLVKLPIVTSKPFDKGKAYDVVYANYGAKKDFEGKDFKGKIALIERGGGLDFMTKITHATNAGVVGIVIFNDQEKRGNFLIPYRELPVGIISKVDGERIKNTSSQLTFNQSFEVVDSQGGNRMLEQSSWGVTAEGAIKPDVTASGFEIYSSTYNNQYQTMSGTSMASPHVAGLMTMLQSHLAEKYKGMNLDSKKLLELSKNILMSSATALYSEEDKAFYSPRQQGAGVVDAEKAIQAQYYITGNDGKAKINLKRMGDKFDITVTIHKLVEGVKELYYQANVATEQVNKGKFALKPQALLDTNWQKVILRDKETQVRFTIDASQFSQKLKEQMANGYFLEGFVRFKEAKDSNQELMSIPFVGFNGDFANLQALETPIYKTLSKGSFYYKPNDTTHKDQLEYNESAPFESNNYTALLTQSASWGYVDYVKNGGELELAPESPKRIILGTFENKVEDKTIHLLERDAANNPYFAISPNKDGNRDEITPQATFLRNVKDISAQVLDQNGNVIWQSKVLPSYRKNFHNNPKQSDGHYRMDALQWSGLDKDGKVVADGFYTYRLRYTPVAEGANSQESDFKVQVSTKSPNLPSRAQFDETNRTLSLAMPKESSYVPTYRLQLVLSHVVKDEEYGDETSYHYFHIDQEGKVTLPKTVKIGESEVAVDPKALTLVVEDKAGNFATVKLSDLLNKAVVSEKENAIVISNSFKYFDNLKKEPMFISKKEKVVNKNLEEIILVKPQTTVTTQSLSKEITKSGNEKVLTSTNNNSSRVAKIISPKHNGDSVNHTLPSTSDRATNGLFVGTLALLSSLLLYLKPKKTKNNSK

SEQ ID NO: 66 - protein, 690 aa, Streptococcus agalactiae 2603
MKKKIILKSSVLGLVAGTSIMFSSVFADQVGVQVIGVNDFHGALDNTGTANMPDGKVANAGTAAQLDAYMDDAQKDFKQTNPNGESIRVQAGDMVGASPANSGLLQDEPTVKNFNAMNVEYGTLGNHEFDEGLAEYNRIVTGKAPAPDSNINNITKSYPHEAAKQEIVVANVIDKVNKQIPYNWKPYAIKNIPVNNKSVNVGFIGIVTKDIPNLVLRKNYEQYEFLDEAETIVKYAKELQAKNVKAIVVLAHVPATSKNDIAEGEAAEMMKKVNQLFPENSVDIVFAGHNHQYTNGLVGKTRIVQALSQGKAYADVRGVLDTDTQDFIETPSAKVIAVAPGKKTGSADIQAIVDQANTIVKQVTEAKIGTAEVSVMITRSVDQDNVSPVGSLITEAQLAIARKSWPDIDFAMTNNGGIRADLLIKPDGTITWGAAQAVQPFGNILQVVEITGRDLYKALNEQYDQKQNFFLQIAGLRYTYTDNKEGGEETPFKVVKAYKSNGEEINPDAKYKLVINDFLFGGGDGFASFRNAKLLGAINPDTEVFMAYITDLEKAGKKVSVPNNKPKIYVTMKMVNETITQNDGTHSIIKKLYLDRQGNIVAQEIVSDTLNQTKSKSTKINPVTTIHKKQLHQFTAINPMRNYGKPSNSTTVKSKQLPKTNSEYGQSFLMSVFGVGLIGIALNTKKKHMK

SEQ ID NO: 67 - protein, 877 aa, Streptococcus agalactiae 2603
MKGQKIIALAGLVLSCHFALTACHTQEHKNSHHIKTKQVAKKKANKKKVSVKESHKKRKGVAGVDFPTDDGFLLTKDSKILSHTDSGIVVAHGNHSHFIFYSDLKGSKFSYLIPNANTKTNKNQAVRNFKAGAVAVNTLNDGYVFNPADIVSEDANGYVVRHGDHFHYIPKASLSQQKQVQASRAVSRLGNQSNSHYRVNSSKIAGLHYPTSNGFLFNGRGIKGTTPTGILVEHHNHLHFISFADLRKGGWGSIADRYQPQKKADSKKQSPSSKKPRTENTLPKDIKDKLAYLARELHLDISRIRVLKTLNGEIGFEYPHDDHTHVIMAKDIDLSKPIPNPHHDDEDHHKGHHHDESDHKHEEHEHTKSNKLSDEDQKKLIYLAEKLGLNPNQIEVLTSEDGSIIFKYPHDDHSHTIASKDIEIGKPIPDGHHDHSHAKDKVGMATLKQIGFDDEIIQDILHADAPTPFPSNETNPEKMRQWLATVTKINIGQRTNPFQRFGLSLMPNIEVLGIGFTPINDMTPVLQFKKLKQLWMTNTGITDYSFLDKMPLLEGLDISQNGIKDLSFLTKYKQLSLIAAANNGITSLKPLAELPNLQFLVLSHNNISDLTPLSNLTKLQELYLDHNNVKNLSALSGKKDLKVLDLSNNKSADLSTLKTTSLETLLLNETNTSNLSFLKQNPKVSNLTINNAKLASLDGIEESDEIVKVEAEGNQIKSLVLKNKQGSLKFLNVTNNQLTSLEGVNNYTSLETLSVSKNKLESLDIKTPNKTVTNLDFSHNNVPTSQLKLNEKNIPEAVAKNFPAVVEGSMVGNGSLAEKAAMASKEDKQVSDNTNHQKNTEKSAQANADSKKENPKTHDEHHDHEETDHAHVGHHHH

SEQ ID NO: 68 - DNA, 36 bp, Artificial Sequence
  Primer
ctgtacttccagggcaataccaatgttttaggggaa

SEQ ID NO: 69 - DNA, 36 bp, Artificial Sequence
  Primer
aattaagtcgcgttattttccactgacagttaactc

SEQ ID NO: 70 - DNA, 36 bp, Artificial Sequence
  Primer
ctgtacttccagggcaccatagtaaaaccagtggac

SEQ ID NO: 71 - DNA, 36 bp, Artificial Sequence
  Primer
aattaagtcgcgttatccattaccaagctgtaaatt

SEQ ID NO: 72 - DNA, 36 bp, Artificial Sequence
  Primer
ctgtacttccagggccaaacattacagccaagtgat

SEQ ID NO: 73 - DNA, 36 bp, Artificial Sequence
  Primer
aattaagtcgcgttatcctttcccacctgtcatagg

SEQ ID NO: 74 - DNA, 36 bp, Artificial Sequence; identical to SEQ ID NO: 68
  Primer

SEQ ID NO: 75 - DNA, 36 bp, Artificial Sequence
  Primer
aattaagtcgcgttatttaccgctaacagttaactc

SEQ ID NO: 76 - DNA, 40 bp, Artificial Sequence
  Primer
ctgtacttccagggctccataataaaaactataaataaag

SEQ ID NO: 77 - DNA, 36 bp, Artificial Sequence
  Primer
aattaagtcgcgttatccgttgccaagatgtaaatt

SEQ ID NO: 78 - DNA, 36 bp, Artificial Sequence
  Primer
ctgtacttccagggccaaacattgcaaccaagtgat

SEQ ID NO: 79 - DNA, 34 bp, Artificial Sequence
  Primer
aattaagtcgcgttatcctttcccacctgtcatc

(19) United States
(12) Patent Application Publication
(10) Pub. No.: US 2013/0216568 A1
(43) Pub. Date: Aug. 22, 2013

(54) IMMUNOGENIC PROTEINS AND COMPOSITIONS

(71) Applicants: Domenico Maione, Siena (IT); Cira Daniela Rinaudo, Castelnuovo Berardenga (IT)

(72) Inventors: Domenico Maione, Siena (IT); Cira Daniela Rinaudo, Castelnuovo Berardenga (IT)

(21) Appl. No.: 13/580,724

(22) PCT Filed: Feb. 28, 2011
(86) PCT No.: PCT/IB11/00562

§ 371 Date: Nov. 28, 2012

(30) Foreign Application Priority Data
Feb. 26, 2010 (GB) 1003333.0

(51) Int. Cl. A61K 39/09; C07K 14/315; A61K 39/385

(52) U.S. Cl. CPC A61K 39/092; A61K 39/385; C07K 14/315
USPC 424/190.1; 530/350; 530/395; 536/23.7; 435/252.3; 424/244.1

(57) ABSTRACT

The invention provides proteins and compositions for the treatment and prevention of Streptococcus agalactiae (Group B streptococcus; GBS).

### TECHNICAL FIELD

The invention provides proteins and compositions for the treatment and prevention of Streptococcus agalactiae (Group B streptococcus; GBS).

### BACKGROUND ART

The Gram-positive bacterium Streptococcus agalactiae (or “group B streptococcus”, abbreviated to “GBS”) causes serious disease, bacteremia and meningitis, in immunocompromised individuals and in neonates. There are two types of neonatal infection. The first (early onset, usually within 5 days of birth) is manifested by bacteremia and pneumonia. It is contracted vertically as a baby passes through the birth canal. GBS colonises the vagina of about 25% of young women, and approximately 1% of infants born via a vaginal birth to colonised mothers will become infected. Mortality is between 50-70%. The second is a meningitis that occurs 10 to 60 days after birth. If pregnant women are vaccinated with type III capsule so that the infants are passively immunised, the incidence of the late onset meningitis is reduced but is not entirely eliminated.

The “B” in “GBS” refers to the Lancefield classification, which is based on the antigenicity of a carbohydrate which is soluble in dilute acid and called the C carbohydrate. Lancefield identified 13 types of C carbohydrate, designated A to O, that could be serologically differentiated. The organisms that most commonly infect humans are found in groups A, B, D, and G. Within group B, strains can be divided into 10 serotypes (Ia, Ib, II, III, IV, V, VI, VII, VII and XI) based on the structure of their polysaccharide capsule.

Investigations have been conducted into the development of protein-based and polysaccharide-based vaccines against GBS but currently, no GBS vaccine is commercially available. There therefore remains a need for effective vaccines against S. agalactiae infection.

It is an object of the invention to provide proteins and immunogenic compositions which can be used in the development of such vaccines.

### DISCLOSURE OF THE INVENTION

Pilus structures in GBS are considered to be interesting vaccine candidates. GBS has three pilus variants, each encoded by a distinct pathogenicity island, PI-1, PI-2a and PI-2b [1, 2]. Each pathogenicity island consists of 5 genes coding for: the pilus backbone protein (BP); 2 ancillary proteins (AP1 and AP2); and 2 sortase proteins that are involved in the assembly of the pili.

All GBS strains carry at least one of these 3 pathogenicity islands and the sequences of the pilus structural proteins (BP, AP1 and AP2) encoded by these pathogenicity islands are generally well conserved. The sequence of ancillary protein 1 (AP1) encoded by pathogenicity island 2a (AP1-2a), also referred to herein as GBS67, varies between GBS strains. At least 2 families of the GBS67 protein exist.

The original ‘GBS67’ (SAG1408) sequence was annotated in reference 147 as a cell wall surface anchor family protein (see GI: 22534437). The amino acid sequence of full length GBS67 as found in the 2603 strain is given as SEQ ID NO: 1 herein. GBS strains CJB111, 515 and NEM316 express GBS67 sequences which belong to the same family as the GBS67 sequence from the 2603 strain. The amino acid sequences of full-length GBS67 as found in the CJB111, 515 and NEM316 strains are given as SEQ ID NOS: 9, 13 and 17 herein.

A variant of GBS67 (SAI1512) exists in strain H36B. This variant ‘GBS67’ (SAG1408) sequence was annotated in reference 3 as a cell wall surface anchor family protein (see GI: 77405751). The amino acid sequence of full length GBS67 as found in the H36B strain is given as SEQ ID NO: 5 herein. GBS strains DK21 and CJB110 express GBS67 sequences which belong to the same family as the GBS67 sequence from the H36B strain. The amino acid sequences of full-length GBS67 as found in the DK21 and CJB110 strains are given as SEQ ID NOS: 21 and 25 herein.

As shown herein, serum raised against the amino acid sequence of full-length GBS67 as found in the 2603 strain and related strains is active against strains of GBS that express the amino acid sequence of full-length GBS67 as found in the H36B strain and related strains, and vice versa. Full-length GBS67 thus provides cross-protection against GBS strains expressing GBS67 variants from either of the two families.

The inventors have now succeeded in identifying fragments of the full-length GBS67 sequences from both the 2603 strain of GBS and the H36B strain of GBS that contain epitopes responsible for cross-protection.

A fragment of the GBS67 sequence as found in the 2603 strain that contains epitopes responsible for cross-protection is given as SEQ ID NO:3 herein. The amino acid sequence of SEQ ID NO:3 is a 398 amino acid fragment located at amino acids 218-615 of the GBS67 sequence from the 2603 strain given in SEQ ID NO:1.

A fragment of the GBS67 sequence as found in the 2603 strain that contains epitopes responsible for cross-protection is given as SEQ ID NO:4 herein. The amino acid sequence of SEQ ID NO:4 is a 251 amino acid fragment located at amino acids 616-866 of the GBS67 sequence from the 2603 strain given in SEQ ID NO:1.

A fragment of the GBS67 sequence as found in the H36B strain that contains epitopes responsible for cross-protection is given as SEQ ID NO:7 herein. The amino acid sequence of SEQ ID NO:7 is a 393 amino acid fragment located at amino acids 218-610 of the GBS67 sequence from the H36B strain given in SEQ ID NO:5.

A fragment of the GBS67 sequence as found in the H36B strain that contains epitopes responsible for cross-protection is given as SEQ ID NO:8 herein. The amino acid sequence of SEQ ID NO:8 is a 251 amino acid fragment located at amino acids 611-861 of the GBS67 sequence from the H36B strain given in SEQ ID NO:5.

Corresponding fragments have also been identified in GBS strains expressing GBS67 from the same family as GBS67 from GBS strain 2603, i.e. GBS strains CJB111, 515 and NEM316, and in GBS strains expressing GBS67 from the same family as GBS strain H36B, i.e. DK21 and CJB110.

A fragment of the GBS67 sequence as found in the CJB111 strain that contains epitopes responsible for cross-protection is given as SEQ ID NO:11 herein. The amino acid sequence of SEQ ID NO:11 is a 398 amino acid fragment located at amino acids 218-615 of the GBS67 sequence from the CJB111 strain given in SEQ ID NO:9.

A fragment of the GBS67 sequence as found in the CJB111 strain that contains epitopes responsible for cross-protection is given as SEQ ID NO:12 herein. The amino acid sequence of SEQ ID NO:12 is a 251 amino acid fragment located at amino acids 616-866 of the GBS67 sequence from the CJB111 strain given in SEQ ID NO:9.

A fragment of the GBS67 sequence as found in the 515 strain that contains epitopes responsible for cross-protection is given as SEQ ID NO:15 herein. The amino acid sequence of SEQ ID NO:15 is a 398 amino acid fragment located at amino acids 218-615 of the GBS67 sequence from the 515 strain given in SEQ ID NO:13.

A fragment of the GBS67 sequence as found in the 515 strain that contains epitopes responsible for cross-protection is given as SEQ ID NO:16 herein. The amino acid sequence of SEQ ID NO:16 is a 251 amino acid fragment located at amino acids 616-866 of the GBS67 sequence from the 515 strain given in SEQ ID NO:13.

A fragment of the GBS67 sequence as found in the NEM316 strain that contains epitopes responsible for cross-protection is given as SEQ ID NO:19 herein. The amino acid sequence of SEQ ID NO:19 is a 398 amino acid fragment located at amino acids 218-615 of the GBS67 sequence from the NEM316 strain given in SEQ ID NO:17.

A fragment of the GBS67 sequence as found in the NEM316 strain that contains epitopes responsible for cross-protection is given as SEQ ID NO:20 herein. The amino acid sequence of SEQ ID NO:20 is a 251 amino acid fragment located at amino acids 616-866 of the GBS67 sequence from the NEM316 strain given in SEQ ID NO:17.

A fragment of the GBS67 sequence as found in the DK21 strain that contains epitopes responsible for cross-protection is given as SEQ ID NO:23 herein. The amino acid sequence of SEQ ID NO:23 is a 393 amino acid fragment located at amino acids 218-610 of the GBS67 sequence from the DK21 strain given in SEQ ID NO:21.

A fragment of the GBS67 sequence as found in the DK21 strain that contains epitopes responsible for cross-protection is given as SEQ ID NO:24 herein. The amino acid sequence of SEQ ID NO:24 is a 251 amino acid fragment located at amino acids 611-861 of the GBS67 sequence from the DK21 strain given in SEQ ID NO:21.

A fragment of the GBS67 sequence as found in the CJB110 strain that contains epitopes responsible for cross-protection is given as SEQ ID NO:27 herein. The amino acid sequence of SEQ ID NO:27 is a 393 amino acid fragment located at amino acids 218-610 of the GBS67 sequence from the CJB110 strain given in SEQ ID NO:25.

A fragment of the GBS67 sequence as found in the CJB110 strain that contains epitopes responsible for cross-protection is given as SEQ ID NO:28 herein. The amino acid sequence of SEQ ID NO:28 is a 251 amino acid fragment located at amino acids 611-861 of the GBS67 sequence from the CJB110 strain given in SEQ ID NO:25.

### GBS67 Polypeptides

These fragments of GBS67 and epitopes from these fragments may be used in place of full-length GBS67 in immunogenic compositions to treat or prevent GBS.

### GBS67 2603

According to one aspect of the invention, therefore, a polypeptide is provided comprising or consisting of:
i) a fragment of at least t contiguous amino acids from SEQ ID NO:1, wherein said fragment comprises an epitope from the amino acid sequence of SEQ ID NO:3 and/or SEQ ID NO:4;
ii) a fragment of at least t contiguous amino acids from an amino acid sequence having at least a % identity to SEQ ID NO:1, wherein said fragment comprises an epitope having at least b % identity to an epitope from the amino acid sequence of SEQ ID NO:3 and/or SEQ ID NO:4;

The polypeptide of this aspect of the invention may comprise or consist of:
i) a fragment of at least t contiguous amino acids from SEQ ID NO:1, wherein said fragment comprises the amino acid sequence of SEQ ID NO:3 and/or SEQ ID NO:4;
ii) a fragment of at least t contiguous amino acids from an amino acid sequence having a % identity to SEQ ID NO:1, wherein said fragment comprises an amino acid sequence having at least b % identity to SEQ ID NO:3 and/or SEQ ID NO:4.

The polypeptide of this aspect of the invention may comprise or consist of a fragment of at least t contiguous amino acids from SEQ ID NO:1 comprising the amino acid sequence of SEQ ID NO:3 and/or SEQ ID NO:4.

### GBS67 H36B

According to another aspect of the invention, a polypeptide is provided comprising or consisting of:
i) a fragment of at least u contiguous amino acids from SEQ ID NO:5, wherein said fragment comprises an epitope from the amino acid sequence of SEQ ID NO:7 and/or SEQ ID NO:8;
ii) a fragment of at least u contiguous amino acids from an amino acid sequence having at least c % identity to SEQ ID NO:5, wherein said fragment comprises an epitope having at least d % identity to an epitope from the amino acid sequence of SEQ ID NO:7 and/or SEQ ID NO:8;

The polypeptide of this aspect of the invention may comprise or consist of:
i) a fragment of at least u contiguous amino acids from SEQ ID NO:5, wherein said fragment comprises the amino acid sequence of SEQ ID NO:7 and/or SEQ ID NO:8;
ii) a fragment of at least u contiguous amino acids from an amino acid sequence having c % identity to SEQ ID NO:5, wherein said fragment comprises an amino acid sequence having at least d % identity to SEQ ID NO:7 and/or SEQ ID NO:8.

The polypeptide of this aspect of the invention may comprise or consist of a fragment of at least u contiguous amino acids from SEQ ID NO:5 comprising the amino acid sequence of SEQ ID NO:7 and/or SEQ ID NO:8.

### GBS67 CJB111

According to another aspect of the invention, a polypeptide is provided comprising or consisting of:
i) a fragment of at least v contiguous amino acids from SEQ ID NO:9, wherein said fragment comprises an epitope from the amino acid sequence of SEQ ID NO:11 and/or SEQ ID NO:12;
ii) a fragment of at least v contiguous amino acids from an amino acid sequence having at least e % identity to SEQ ID NO:9, wherein said fragment comprises an epitope having at least f % identity to an epitope from the amino acid sequence of SEQ ID NO:11 and/or SEQ ID NO:12;

The polypeptide of this aspect of the invention may comprise or consist of:
i) a fragment of at least v contiguous amino acids from SEQ ID NO:9, wherein said fragment comprises the amino acid sequence of SEQ ID NO:11 and/or SEQ ID NO:12;
ii) a fragment of at least v contiguous amino acids from an amino acid sequence having e % identity to SEQ ID NO:9, wherein said fragment comprises an amino acid sequence having at least f % identity to SEQ ID NO:11 and/or SEQ ID NO:12.

The polypeptide of this aspect of the invention may comprise or consist of a fragment of at least v contiguous amino acids from SEQ ID NO:9 comprising the amino acid sequence of SEQ ID NO:11 and/or SEQ ID NO:12.

### GBS67 515

According to another aspect of the invention, a polypeptide is provided comprising or consisting of:
i) a fragment of at least w contiguous amino acids from SEQ ID NO:13, wherein said fragment comprises an epitope from the amino acid sequence of SEQ ID NO:15 and/or SEQ ID NO:16;
ii) a fragment of at least w contiguous amino acids from an amino acid sequence having at least g % identity to SEQ ID NO:13, wherein said fragment comprises an epitope having at least h % identity to an epitope from the amino acid sequence of SEQ ID NO:15 and/or SEQ ID NO:16;

The polypeptide of this aspect of the invention may comprise or consist of:
i) a fragment of at least w contiguous amino acids from SEQ ID NO:13, wherein said fragment comprises the amino acid sequence of SEQ ID NO:15 and/or SEQ ID NO:16;
ii) a fragment of at least w contiguous amino acids from an amino acid sequence having g % identity to SEQ ID NO:13, wherein said fragment comprises an amino acid sequence having at least h % identity to SEQ ID NO:15 and/or SEQ ID NO:16.

The polypeptide of this aspect of the invention may comprise or consist of a fragment of at least w contiguous amino acids from SEQ ID NO:13 comprising the amino acid sequence of SEQ ID NO:15 and/or SEQ ID NO:16.

### GBS67 NEM316

According to another aspect of the invention, a polypeptide is provided comprising or consisting of:
i) a fragment of at least x contiguous amino acids from SEQ ID NO:17, wherein said fragment comprises an epitope from the amino acid sequence of SEQ ID NO:19 and/or SEQ ID NO:20;
ii) a fragment of at least x contiguous amino acids from an amino acid sequence having at least i % identity to SEQ ID NO:17, wherein said fragment comprises an epitope having at least j % identity to an epitope from the amino acid sequence of SEQ ID NO:19 and/or SEQ ID NO:20;

The polypeptide of this aspect of the invention may comprise or consist of:
i) a fragment of at least x contiguous amino acids from SEQ ID NO:17, wherein said fragment comprises the amino acid sequence of SEQ ID NO:19 and/or SEQ ID NO:20;
ii) a fragment of at least x contiguous amino acids from an amino acid sequence having i % identity to SEQ ID NO:17, wherein said fragment comprises an amino acid sequence having at least j % identity to SEQ ID NO:19 and/or SEQ ID NO:20.

The polypeptide of this aspect of the invention may comprise or consist of a fragment of at least x contiguous amino acids from SEQ ID NO:17 comprising the amino acid sequence of SEQ ID NO:19 and/or SEQ ID NO:20.

### GBS67 DK21

According to another aspect of the invention, a polypeptide is provided comprising or consisting of:
i) a fragment of at least y contiguous amino acids from SEQ ID NO:21, wherein said fragment comprises an epitope from the amino acid sequence of SEQ ID NO:23 and/or SEQ ID NO:24;
ii) a fragment of at least y contiguous amino acids from an amino acid sequence having at least k % identity to SEQ ID NO:21, wherein said fragment comprises an epitope having at least 1% identity to an epitope from the amino acid sequence of SEQ ID NO:23 and/or SEQ ID NO:24;

The polypeptide of this aspect of the invention may comprise or consist of:
i) a fragment of at least y contiguous amino acids from SEQ ID NO:21, wherein said fragment comprises the amino acid sequence of SEQ ID NO:23 and/or SEQ ID NO:24;
ii) a fragment of at least y contiguous amino acids from an amino acid sequence having k % identity to SEQ ID NO:21, wherein said fragment comprises an amino acid sequence having at least 1% identity to SEQ ID NO:23 and/or SEQ ID NO:24.

The polypeptide of this aspect of the invention may comprise or consist of a fragment of at least y contiguous amino acids from SEQ ID NO:21 comprising the amino acid sequence of SEQ ID NO:23 and/or SEQ ID NO:24.

### GBS67 CJB110

According to another aspect of the invention, a polypeptide is provided comprising or consisting of:
i) a fragment of at least z contiguous amino acids from SEQ ID NO:25, wherein said fragment comprises an epitope from the amino acid sequence of SEQ ID NO:27 and/or SEQ ID NO:28;
ii) a fragment of at least z contiguous amino acids from an amino acid sequence having at least m % identity to SEQ ID NO:25, wherein said fragment comprises an epitope having at least n % identity to an epitope from the amino acid sequence of SEQ ID NO:27 and/or SEQ ID NO:28;

The polypeptide of this aspect of the invention may comprise or consist of:
i) a fragment of at least z contiguous amino acids from SEQ ID NO:25, wherein said fragment comprises the amino acid sequence of SEQ ID NO:27 and/or SEQ ID NO:28;
ii) a fragment of at least z contiguous amino acids from an amino acid sequence having m % identity to SEQ ID NO:25, wherein said fragment comprises an amino acid sequence having at least n % identity to SEQ ID NO:27 and/or SEQ ID NO:28.

The polypeptide of this aspect of the invention may comprise or consist of a fragment of at least z contiguous amino acids from SEQ ID NO:25 comprising the amino acid sequence of SEQ ID NO:27 and/or SEQ ID NO:28.

By “epitope” is meant the part of the polypeptide that is recognised by the immune system and that elicits an immune response. The polypeptides of the invention are capable of inducing cross-protection against strains of GBS expressing variant GBS67 peptides. Thus, the polypeptides of the invention will, when administered to a subject, elicit an antibody response comprising antibodies that bind to the wild-type GBS protein having amino acid sequence SEQ ID NO: 1 (strain 2603) and to the wild-type GBS protein having amino acid sequence SEQ ID NO: 5 (strain H36B). The polypeptides of the invention are thus capable of competing with both SEQ ID NO: 1 and SEQ ID NO:5 for binding to an antibody raised against SEQ ID NO: 1 or SEQ ID NO:5.

The polypeptides of the invention will typically also, when administered to a subject, elicit an antibody response comprising antibodies that bind to the wild-type GBS protein having amino acid sequence SEQ ID NO: 9 (strain CJB111), the wild-type GBS protein having amino acid sequence SEQ ID NO: 13 (strain 515), the wild-type GBS protein having amino acid sequence SEQ ID NO: 17 (strain NEM316), the wild-type GBS protein having amino acid sequence SEQ ID NO: 21 (strain DK21), and the wild-type GBS protein having amino acid sequence SEQ ID NO: 25 (strain CJB 110). The polypeptides of the invention are thus also capable of competing with these wild-type GBS proteins having SEQ ID NOs:9, 13, 17, 21 or 25 for binding to an antibody raised against these proteins.

Antibodies can readily be generated against the polypeptides of the invention using standard immunisation methods and the ability of these antibodies to bind to the wild-type GBS proteins of SEQ ID NOs: 1, 5, 9, 13, 17, 21 and 25 can be assessed using standard assays such as ELISA assays.

Similarly, the ability of polypeptides to compete with antibodies raised against the wild-type GBS proteins can be readily determined using competition assay techniques known in the art, including equilibrium methods such as ELISA, kinetic methods such as BIACORE® and by flow cytometry methods. A polypeptide that competes with wild-type GBS proteins of SEQ ID NOs: 1, 5, 9, 13, 17, 21 and 25 for binding to an antibody against one of these wild-type GBS proteins will cause a reduction in the observed total binding of the wild-type GBS protein to the antibody, compared to when the polypeptide is not present. Typically, this reduction in binding is 10% or greater, 20% or greater, 30% or greater, 40% or greater, 60% or greater, for example a reduction in binding of 70% or more in the presence of the polypeptide of the invention compared to antibody binding observed for the GBS proteins having SEQ ID NO:1, 5, 9, 13, 17, 21 or 25.

The ability of the polypeptides of the invention to induce cross-protection against strains of GBS expressing variant GBS67 proteins can also be confirmed in animal models, such as the maternal immunization models described in the examples in which female mice are immunized with the polypeptides and their pups are challenged with GBS strains expressing variant GBS67 proteins.

The value of a is at least 75 e.g. 80, 85, 90, 92, 94, 95, 96, 97, 98, 99 or more. The value of b is at least 75 e.g. 80, 85, 90, 92, 94, 95, 96, 97, 98, 99 or more. The value of c is at least 75 e.g. 80, 85, 90, 92, 94, 95, 96, 97, 98, 99 or more. The value of d is at least 75 e.g. 80, 85, 90, 92, 94, 95, 96, 97, 98, 99 or more. The value of e is at least 75 e.g. 80, 85, 90, 92, 94, 95, 96, 97, 98, 99 or more. The value off is at least 75 e.g. 80, 85, 90, 92, 94, 95, 96, 97, 98, 99 or more. The value of g is at least 75 e.g. 80, 85, 90, 92, 94, 95, 96, 97, 98, 99 or more. The value of h is at least 75 e.g. 80, 85, 90, 92, 94, 95, 96, 97, 98, 99 or more. The value of i is at least 75 e.g. 80, 85, 90, 92, 94, 95, 96, 97, 98, 99 or more. The value of j is at least 75 e.g. 80, 85, 90, 92, 94, 95, 96, 97, 98, 99 or more. The value of k is at least 75 e.g. 80, 85, 90, 92, 94, 95, 96, 97, 98, 99 or more. The value of l is at least 75 e.g. 80, 85, 90, 92, 94, 95, 96, 97, 98, 99 or more. The value of m is at least 75 e.g. 80, 85, 90, 92, 94, 95, 96, 97, 98, 99 or more. The value of n is at least 75 e.g. 80, 85, 90, 92, 94, 95, 96, 97, 98, 99 or more. Typically, a, b, c, d, e, f g, h, i, j, k, l, m and n are at least 90 e.g. at least 95.

The value of t is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400. The full-length GBS67 sequence from the 2603 strain as shown in SEQ ID NO:1 is 901 amino acids long. The value of t is thus also less than 901, e.g. less than 850, 800, 750, 700, 650, 600, 550, 500, 450. The value of t may be between 50-600, 100-400, 150-300, 225-275, e.g. 120-150.

The value of u is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400. The full-length GBS67 sequence from the H36B strain as shown in SEQ ID NO:5 is 896 amino acids long. The value of u is thus also less than 896, e.g. less than 860, 850, 800, 750, 700, 650, 600, 550, 500, 450. The value of u may be between 50-600, 100-400, 150-300, 225-275, e.g. 120-150.

The value of v is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400. The full-length GBS67 sequence from the CJB 111 strain as shown in SEQ ID NO:9 is 901 amino acids long. The value of v is thus also less than 901, e.g. less than 860, 850, 800, 750, 700, 650, 600, 550, 500, 450. The value of v may be between 50-600, 100-400, 150-300, 225-275, e.g. 120-150.

The value of w is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400. The full-length GBS67 sequence from the 515 strain as shown in SEQ ID NO:13 is 901 amino acids long. The value of w is thus also less than 901, e.g. less than 860, 850, 800, 750, 700, 650, 600, 550, 500, 450. The value of w may be between 50-600, 100-400, 150-300, 225-275, e.g. 120-150.

The value of x is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400. The full-length GBS67 sequence from the NEM316 strain as shown in SEQ ID NO:17 is 901 amino acids long. The value of x is thus also less than 901, e.g. less than 860, 850, 800, 750, 700, 650, 600, 550, 500, 450. The value of w may be between 50-600, 100-400, 150-300, 225-275, e.g. 120-150.

The value of y is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400. The full-length GBS67 sequence from the DK21 strain as shown in SEQ ID NO:21 is 896 amino acids long. The value of y is thus also less than 896, e.g. less than 860, 850, 800, 750, 700, 650, 600, 550, 500, 450. The value of y may be between 50-600, 100-400, 150-300, 225-275, e.g. 120-150.

The value of z is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400. The full-length GBS67 sequence from the CJB110 strain as shown in SEQ ID NO:25 is 896 amino acids long. The value of z is thus also less than 896, e.g. less than 860, 850, 800, 750, 700, 650, 600, 550, 500, 450. The value of z may be between 50-600, 100-400, 150-300, 225-275, e.g. 120-150.

The polypeptides of the invention may, compared with fragments of SEQ ID NOs: 1, 5, 9, 13, 17, 21 and 25 include one or more (e.g. 1, 2, 3, 4, 5, 6, 7, 8, 9, 10, etc.) conservative amino acid replacements i.e. replacements of one amino acid with another which has a related side chain. These conservative amino acid replacements may be located within the regions of SEQ ID NOs: 1, 5, 9, 13, 17, 21 and 25 corresponding to SEQ ID NOs: 3 and 4, 7 and 8, 11 and 12, 15 and 16, 19 and 20, 23 and 24, or 27 and 28 respectively. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity.

The polypeptides of the invention may have one or more (e.g. 1, 2, 3, 4, 5, 6, 7, 8, 9, 10, etc.) single amino acid deletions relative to fragments of SEQ ID NOs: 1, 5, 9, 13, 17, 21 and 25. The polypeptides may also include one or more (e.g. 1, 2, 3, 4, 5, 6, 7, 8, 9, 10, etc.) insertions (e.g. each of 1, 2, 3, 4 or 5 amino acids) relative to fragments of SEQ ID NOs: 1, 5, 9, 13, 17, 21 and 25. These deletions and insertions may be located within the regions of SEQ ID NOs: 1, 5, 9, 13, 17, 21 and 25 corresponding to SEQ ID NOs: 3 and 4, 7 and 8, 11 and 12, 15 and 16, 19 and 20, 23 and 24, or 27 and 28, respectively.

A polypeptide of the invention may comprise an amino acid sequence that:
(a) is identical (i.e. 100% identical) to a fragment of SEQ ID NO: 1, SEQ ID NO: 5, SEQ ID NO:9, SEQ ID NO:13, SEQ ID NO:17, SEQ ID NO:21 or SEQ ID NO:25;
(b) shares sequence identity with a fragment of SEQ ID NO: 1, SEQ ID NO: 5, SEQ ID NO:9, SEQ ID NO:13, SEQ ID NO:17, SEQ ID NO:21 or SEQ ID NO:25;
(c) has 1, 2, 3, 4, 5, 6, 7, 8, 9 or 10 (or more) single amino acid alterations (deletions, insertions, substitutions), which may be at separate locations or may be contiguous, as compared to the sequences of (a) or (b); and
(d) when aligned with a fragment of SEQ ID NO: 1, SEQ ID NO: 5, SEQ ID NO:9, SEQ ID NO:13, SEQ ID NO:17, SEQ ID NO:21 or SEQ ID NO:25 using a pairwise alignment algorithm, each moving window of x amino acids from N-terminus to C-terminus (such that for an alignment that extends to p amino acids, where p>x, there are p−x+1 such windows) has at least x-y identical aligned amino acids, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if x·y is not an integer then it is rounded up to the nearest integer. The preferred pairwise alignment algorithm is the Needleman-Wunsch global alignment algorithm [4], using default parameters (e.g. with Gap opening penalty=10.0, and with Gap extension penalty=0.5, using the EBLOSUM62 scoring matrix). This algorithm is conveniently implemented in the needle tool in the EMBOSS package [5].

The polypeptides of the invention may be provided in the form of a hybrid polypeptide. The hybrid polypeptide may comprise additional GBS or non-GBS polypeptide sequences.

The invention also provides a nucleic acid comprising a nucleotide sequence encoding a polypeptide or a hybrid polypeptide of the invention.

The invention also provides an immunogenic composition comprising a polypeptide, a hybrid polypeptide or a nucleic acid of the invention. Such an immunogenic composition may be used in methods of treating or preventing diseases or conditions associated with GBS.

The invention also provides a cell (typically a bacterium) which expresses a polypeptide or a hybrid polypeptide of the invention.

### Hybrid Polypeptides

The polypeptides of the invention can be expressed in combination with other polypeptides as a single polypeptide chain (a ‘hybrid’ polypeptide or ‘chimera’). Hybrid polypeptides offer two main advantages: first, a polypeptide that may be unstable or poorly expressed on its own can be assisted by adding a suitable hybrid partner that overcomes the problem; second, commercial manufacture is simplified as only one expression and purification need to be employed in order to produce two polypeptides which are both antigenically useful.

Hybrid polypeptides can include sequences from other GBS antigens and/or from other non-GBS antigens. Usually, the hybrid polypeptides include sequences from other GBS sequences, such as other pilus subunits. These other GBS sequence may be to the N-terminus or to the C-terminus of the GBS67 polypeptides. Different hybrid polypeptides may be mixed together in a single formulation.

Hybrid polypeptides may be represented by the formula NH₂-A-{-X-L-}ₙ-B—COOH.

X is a GBS67 polypeptide of the invention, as discussed above. If a —X— moiety has a leader peptide sequence in its wild-type form, this may be included or omitted in the hybrid protein. In some embodiments, the leader peptides will be deleted except for that of the —X— moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of {—X-L-}, linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s) -L- will typically be short (e.g. 20 or fewer amino acids i.e. 20, 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. H is where n=3, 4, 5, 6, 7, 8, 9, 10 or more). Other suitable linker amino acid sequences will be apparent to those skilled in the art. Useful linkers are GSGS (SEQ ID NO:29), GSGGGG (SEQ ID NO: 30) or GSGSGGGG (SEQ ID NO: 31), with the Gly-Ser dipeptide being formed from a BamHI restriction site, thus aiding cloning and manipulation, and the (Gly)₄ tetrapeptide being a typical poly-glycine linker. Other suitable linkers, particularly for use as the final Lₙ are a Leu-Glu dipeptide or Gly-Ser. Linkers will usually contain at least one glycine residue to facilitate structural flexibility e.g. a -L- moiety may contain 1, 2, 3, 4, 5, 6, 7, 8, 9, 10 or more glycine residues. Such glycines may be arranged to include at least two consecutive glycines in a Gly-Gly dipeptide sequence, or a longer oligo-Gly sequence i.e. Glyₙ where n=2, 3, 4, 5, 6, 7, 8, 9, 10 or more.

-A- is an optional N-terminal amino acid sequence. This will typically be short (e.g. 40 or fewer amino acids i.e. 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. Hisₙ where n=3, 4, 5, 6, 7, 8, 9, 10 or more). Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine e.g. Met-Ala-Ser, or a single Met residue. In a nascent polypeptide the -A- moiety can provide the polypeptide's N-terminal methionine (formyl-methionine, fMet, in bacteria). One or more amino acids may be cleaved from the N-terminus of a nascent -A- moiety, however, such that the -A- moiety in a mature polypeptide of the invention does not necessarily include a N-terminal methionine.

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminal amino acid sequences will be apparent to those skilled in the art, such as a glutathione-S-transferase, thioredoxin, 14 kDa fragment of S. aureus protein A, a biotinylated peptide, a maltose-binding protein, an enterokinase flag, etc.

It is preferred that -A-, —B— and -L- sequences do not include a sequence that shares 10 or more contiguous amino acids in common with a human polypeptide sequence.

In some embodiments, a -L- moiety comprises a non-GBS67 antigen. In some embodiments, the -A- moiety comprises a non-GBS67 antigen, and in some the —B— moiety comprises a non-GBS67 antigen.

### Polypeptides

Polypeptides used with the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides<40 amino acids long involves in vitro chemical synthesis [6,7]. Solid-phase peptide synthesis is particularly preferred, such as methods based on tBoc or Fmoc [8] chemistry. Enzymatic synthesis [9] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [10]. Where D-amino acids are included, however, it is preferred to use chemical synthesis. Polypeptides may have covalent modifications at the C-terminus and/or N-terminus.

Polypeptides can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides are preferably provided in purified or substantially purified form i.e. substantially free from other polypeptides (e.g. free from naturally-occurring polypeptides), particularly from other pneumococcal or host cell polypeptides, and are generally at least about 50% pure (by weight), and usually at least about 90% pure i.e. less than about 50%, and more preferably less than about 10% (e.g. 5% or less) of a composition is made up of other expressed polypeptides.

Polypeptides may be attached to a solid support. Polypeptides may comprise a detectable label (e.g. a radioactive or fluorescent label, or a biotin label).

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides can be naturally or non-naturally glycosylated (i.e. the polypeptide has a glycosylation pattern that differs from the glycosylation pattern found in the corresponding naturally occurring polypeptide).

The invention provides a process for producing polypeptides of the invention, comprising culturing a host cell of to the invention under conditions which induce polypeptide expression. Although expression of the polypeptide may take place in a Streptococcus, the invention will usually use a heterologous host for expression. The heterologous host may be prokaryotic (e.g. a bacterium) or eukaryotic. It will usually be E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc.

The invention also provides a process for producing a polypeptide of the invention, wherein the polypeptide is synthesised in part or in whole using chemical means.

The invention also provides a composition comprising two or more polypeptides of the invention.

### Nucleic Acids

The invention also provides a nucleic acid comprising a nucleotide sequence encoding a polypeptide or a hybrid polypeptide of the invention.

For example, the invention provides a nucleic acid comprising a nucleotide sequence encoding a polypeptide comprising or consisting of an amino acid sequence selected from the group consisting of: SEQ ID NO:3, SEQ ID NO:4, SEQ ID NO:7, SEQ ID NO:8, SEQ ID NO:11, SEQ ID NO:12, SEQ ID NO:15, SEQ ID NO:16, SEQ ID NO:19, SEQ ID NO:20, SEQ ID NO:23, SEQ ID NO:24, SEQ ID NO:27 or SEQ ID NO:28.

The invention also provides nucleic acids comprising nucleotide sequences having sequence identity to such nucleotide sequences. Such nucleic acids include those using alternative codons to encode the same amino acid. In particular, nucleic acids may contain alternative codons optimised for expression in specific microorganisms, e.g. E. coli.

The invention also provides nucleic acid which can hybridize to these nucleic acids. Hybridization reactions can be performed under conditions of different “stringency”. Conditions that increase stringency of a hybridization reaction of widely known and published in the art. Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art [e.g. see refs 11 & 222, etc.].

The invention includes nucleic acid comprising sequences complementary to these sequences (e.g. for antisense or probing, or for use as primers).

Nucleic acids according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably provided in purified or substantially purified form i.e. substantially free from other nucleic acids (e.g. free from naturally-occurring nucleic acids), particularly from other GBS or host cell nucleic acids, generally being at least about 50% pure (by weight), and usually at least about 90% pure. Nucleic acids of the invention are preferably GBS nucleic acids.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T).

Nucleic acids of the invention can be used, for example: to produce polypeptides in vitro or in vivo; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid of the invention, wherein the nucleic acid is synthesised in part or in whole using chemical means.

The invention provides vectors comprising nucleotide sequences of the invention (e.g. cloning or expression vectors) and host cells transformed with such vectors.

### Immunogenic Compositions

The polypeptides and hybrid polypeptides of the invention are useful as active ingredients in immunogenic compositions. Such immunogenic compositions may be useful as vaccines. These vaccines may either be prophylactic (i.e. to prevent infection) or therapeutic (i.e. to treat infection), but will typically be prophylactic.

Compositions may thus be pharmaceutically acceptable. They will usually include components in addition to the antigens e.g. they typically include one or more pharmaceutical carrier(s) and/or excipient(s). A thorough discussion of such components is available in reference [217].

Compositions will generally be administered to a mammal in aqueous form. Prior to administration, however, the composition may have been in a non-aqueous form. For instance, although some vaccines are manufactured in aqueous form, then filled and distributed and administered also in aqueous form, other vaccines are lyophilised during manufacture and are reconstituted into an aqueous form at the time of use. Thus a composition of the invention may be dried, such as a lyophilised formulation.

The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred.

To control tonicity, it is preferred to include a physiological salt, such as a sodium salt. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

Immunogenic compositions of the invention may also comprise one or more immunoregulatory agents. Preferably, one or more of the immunoregulatory agents include one or more adjuvants. The adjuvants may include a TH1 adjuvant and/or a TH2 adjuvant, further discussed below.

Adjuvants which may be used in compositions of the invention include, but are not limited to:

### A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. see chapters 8 & 9 of ref. 12], or mixtures of different mineral compounds, with the compounds taking any suitable form (e.g. gel, crystalline, amorphous, etc.), and with adsorption being preferred. The mineral containing compositions may also be formulated as a particle of metal salt.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ [chapter 9 of ref. 12]. The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls [ch. 9 of ref. 12].

The PO₄/Al³⁺ molar ratio of an aluminium phosphate adjuvant will generally be between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, preferably between 5 and 15 mM, and more preferably about 10 mM. The suspensions may also comprise sodium chloride.

In one embodiment, an adjuvant component includes a mixture of both an aluminium hydroxide and an aluminium phosphate. In this case there may be more aluminium phosphate than hydroxide e.g. a weight ratio of at least 2:1 e.g. ≧5:1, ≧6:1, ≧7:1, ≧8:1, ≧9:1, etc.

The concentration of Al in a composition for administration to a patient is preferably less than 10 mg/ml e.g. ≦5 mg/ml, ≦4 mg/ml, ≦3 mg/ml, ≦2 mg/ml, ≦1 mg/ml, etc. A preferred range is between 0.3 and 1 mg/ml. A maximum of <0.85 mg/dose is preferred.

### B. Oil Emulsions

Oil emulsion compositions suitable for use as adjuvants in the invention include squalene-water emulsions, such as MF59 [Chapter 10 of ref. 12; see also ref. 13] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

Various suitable oil-in-water emulsions are known, and they typically include at least one oil and at least one surfactant, with the oil(s) and surfactant(s) being biodegradable (metabolisable) and biocompatible. The oil droplets in the emulsion are generally less than 5 μm in diameter, and advantageously the emulsion comprises oil droplets with a sub-micron diameter, with these small sizes being achieved with a microfluidiser to provide stable emulsions. Droplets with a size less than 220 nm are preferred as they can be subjected to filter sterilization.

The invention can be used with oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoid known as squalene, 2,6,10,15,19,23-hexamethyl-2,6,10,14,18,22-tetracosahexaene. Other preferred oils are the tocopherols (see below).

Oil in water emulsions comprising squalene are particularly preferred. Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Preferred surfactants for including in the emulsion are Tween 80 (polyoxyethylene sorbitan monooleate), Span 85 (sorbitan trioleate), lecithin and Triton X-100. As mentioned above, detergents such as Tween 80 may contribute to the thermal stability seen in the examples below.

Mixtures of surfactants can be used e.g. Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Specific oil-in-water emulsion adjuvants useful with the invention include, but are not limited to:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [14-16], as described in more detail in Chapter 10 of ref. 17 and chapter 12 of ref. 18. The MF59 emulsion advantageously includes citrate ions e.g. 10 mM sodium citrate buffer.
An emulsion comprising squalene, an α-tocopherol, and polysorbate 80. These emulsions may have from 2 to 10% squalene, from 2 to 10% tocopherol and from 0.3 to 3% Tween 80, and the weight ratio of squalene:tocopherol is preferably ≦1 (e.g. 0.90) as this provides a more stable emulsion. Squalene and Tween 80 may be present volume ratio of about 5:2, or at a weight ratio of about 11:5. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm.
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 μg/ml polysorbate 80, 110 μg/ml Triton X-100 and 100 μg/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [19] (0.05-1% Thr-MDP, 5% squalane, 2.5% Pluronic L121 and 0.2% polysorbate 80). It can also be used without the Thr-MDP, as in the “AF” adjuvant [20] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [21]. The emulsion may also include one or more of: alditol; a cryoprotective agent (e.g. a sugar, such as dodecylmaltoside and/or sucrose); and/or an alkylpolyglycoside. Such emulsions may be lyophilized.
An emulsion having from 0.5-50% of an oil, 0.1-10% of a phospholipid, and 0.05-5% of a non-ionic surfactant. As described in reference 22, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 23, produced by addition of aliphatic amine to desacylsaponin via the carboxyl group of glucuronic acid), dimethyldioctadecylammonium bromide and/or N,N-dioctadecyl-N,N-bis(2-hydroxyethyl)propanediamine.
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [24].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [24].
An emulsion in which a saponin (e.g. QuilA or QS21) and a sterol (e.g. a cholesterol) are associated as helical micelles [25].

Antigens and adjuvants in a composition will typically be in admixture at the time of delivery to a patient. The emulsions may be mixed with antigen during manufacture, or extemporaneously, at the time of delivery. Thus the adjuvant and antigen may be kept separately in a packaged or distributed vaccine, ready for final formulation at the time of use. The antigen will generally be in an aqueous form, such that the vaccine is finally prepared by mixing two liquids. The volume ratio of the two liquids for mixing can vary (e.g. between 5:1 and 1:5) but is generally about 1:1.

### C. Saponin Formulations [Chapter 22 of ref. 12]

Saponin formulations may also be used as adjuvants in the invention. Saponins are a heterogeneous group of sterol glycosides and triterpenoid glycosides that are found in the bark, leaves, sterns, roots and even flowers of a wide range of plant species. Saponin from the bark of the Quillaia saponaria Molina tree have been widely studied as adjuvants. Saponin can also be commercially obtained from Smilax ornata (sarsaparilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 26. Saponin formulations may also comprise a sterol, such as cholesterol [27].

Combinations of saponins and cholesterols can be used to form unique particles called immunostimulating complexes (ISCOMs) [chapter 23 of ref 12]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 27-29. Optionally, the ISCOMS may be devoid of additional detergent [30].

A review of the development of saponin based adjuvants can be found in refs. 31 & 32.

### D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in refs. 33-38. Virosomes are discussed further in, for example, ref. 39

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref 40. Such “small particles” of 3dMPL are small enough to be sterile filtered through a 0.22 μm membrane [40]. Other non-toxic LPS derivatives include monophosphoryl lipid A mimics, such as aminoalkyl glucosaminide phosphate derivatives e.g. RC-529 [41,42].

Lipid A derivatives include derivatives of lipid A from Escherichia coli such as OM-174. OM-174 is described for example in refs. 43 & 44.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 45, 46 and 47 disclose possible analog substitutions e.g. replacement of guanosine with 2′-deoxy-7-deazaguanosine. The adjuvant effect of CpG oligonucleotides is further discussed in refs. 48-53.

The CpG sequence may be directed to TLR9, such as the motif GTCGTT or TTCGTT [54]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 55-57. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 54 & 58-60.

A particularly useful adjuvant based around immunostimulatory oligonucleotides is known as IC-31™ [61]. Thus an adjuvant used with the invention may comprise a mixture of (i) an oligonucleotide (e.g. between 15-40 nucleotides) including at least one (and preferably multiple) CpI motifs (i.e. a cytosine linked to an inosine to form a dinucleotide), and (ii) a polycationic polymer, such as an oligopeptide (e.g. between 5-20 amino acids) including at least one (and preferably multiple) Lys-Arg-Lys tripeptide sequence(s). The oligonucleotide may be a deoxynucleotide comprising 26-mer sequence 5′-(IC)₁₃-3′ (SEQ ID NO: 32). The polycationic polymer may be a peptide comprising 11-mer amino acid sequence KLKLLLLLKLK (SEQ ID NO: 33).

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 62 and as parenteral adjuvants in ref 63. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivatives thereof, particularly LT-K63 and LT-R72, as adjuvants can be found in refs. 64-71. A useful CT mutant is or CT-E29H [72]. Numerical reference for amino acid substitutions is preferably based on the alignments of the A and B subunits of ADP-ribosylating toxins set forth in ref. 73, specifically incorporated herein by reference in its entirety.

### F. Human Immunomodulators

Human immunomodulators suitable for use as adjuvants in the invention include cytokines, such as interleukins (e.g. IL-1, IL-2, IL-4, IL-5, IL-6, IL-7, IL-12 [74], etc.) [75], interferons (e.g. interferon-γ), macrophage colony stimulating factor, and tumor necrosis factor. A preferred immunomodulator is IL-12.

### G. Bioadhesives and Mucoadhesives

Bioadhesives and mucoadhesives may also be used as adjuvants in the invention. Suitable bioadhesives include esterified hyaluronic acid microspheres [76] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [77].

### H. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### I. Liposomes (Chapters 13 & 14 of ref 12)

Examples of liposome formulations suitable for use as adjuvants are described in refs. 78-80.

### J. Polyoxyethylene Ether and Polyoxyethylene Ester Formulations

Adjuvants suitable for use in the invention include polyoxyethylene ethers and polyoxyethylene esters [81]. Such formulations further include polyoxyethylene sorbitan ester surfactants in combination with an octoxynol [82] as well as polyoxyethylene alkyl ethers or ester surfactants in combination with at least one additional non-ionic surfactant such as an octoxynol [83]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### K. Polyphosphazene (PCPP)

PCPP formulations are described, for example, in refs. 84 and 85.

### L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### M. Imidazoquinolone Compounds.

Examples of imidazoquinolone compounds suitable for use adjuvants in the invention include Imiquamod and its homologues (e.g. “Resiquimod 3M”), described further in refs. 86 and 87.

The invention may also comprise combinations of aspects of one or more of the adjuvants identified above. For example, the following adjuvant compositions may be used in the invention: (1) a saponin and an oil-in-water emulsion [88]; (2) a saponin (e.g. QS21)+a non-toxic LPS derivative (e.g. 3dMPL) [89]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [90]; (5) combinations of 3dMPL with, for example, QS21 and/or oil-in-water emulsions [91]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL).

Other substances that act as immunostimulating agents are disclosed in chapter 7 of ref 12.

The use of an aluminium hydroxide and/or aluminium phosphate adjuvant is useful, particularly in children, and antigens are generally adsorbed to these salts. Squalene-in-water emulsions are also preferred, particularly in the elderly. Useful adjuvant combinations include combinations of Th1 and Th2 adjuvants such as CpG & alum or resiquimod & alum. A combination of aluminium phosphate and 3dMPL may be used.

The compositions of the invention may elicit both a cell mediated immune response as well as a humoral immune response.

Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs). CD8 T cells are able to recognized or interact with antigens displayed on MHC Class I molecules.

CD4 T cells can express a CD4 co-receptor and are commonly referred to as T helper cells. CD4 T cells are able to recognize antigenic peptides bound to MHC class II molecules. Upon interaction with a MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH 1 cells may secrete one or more of IL-2, IFN-γ, and TNF-β. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response and a TH2 immune response.

A TH1 immune response may include one or more of an increase in CTLs, an increase in one or more of the cytokines associated with a TH1 immune response (such as IL-2, IFN-γ, and TNF-β), an increase in activated macrophages, an increase in NK activity, or an increase in the production of IgG2a. Preferably, the enhanced TH 1 immune response will include an increase in IgG2a production.

A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred TH1 adjuvants for use in the invention.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG1 production.

A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives thereof. Mineral containing compositions, such as aluminium salts are preferred TH2 adjuvants for use in the invention.

A composition may include a combination of a TH1 adjuvant and a TH2 adjuvant. Preferably, such a composition elicits an enhanced TH1 and an enhanced TH2 response, i.e., an increase in the production of both IgG1 and IgG2a production relative to immunization without an adjuvant. Still more preferably, the composition comprising a combination of a TH1 and a TH2 adjuvant elicits an increased TH1 and/or an increased TH2 immune response relative to immunization with a single adjuvant (i.e., relative to immunization with a TH1 adjuvant alone or immunization with a TH2 adjuvant alone).

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

Streptococcal infections can affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

### Nucleic Acid Immunisation

The immunogenic compositions described above include polypeptide antigens from GBS. In all cases, however, the polypeptide (and hybrid polypeptide) antigens can be replaced by nucleic acids (typically DNA) encoding those polypeptides, to give compositions, methods and uses based on nucleic acid immunisation [92 to 99

The nucleic acid encoding the immunogen is expressed in vivo after delivery to a patient and the expressed immunogen then stimulates the immune system. The active ingredient will typically take the form of a nucleic acid vector comprising: (i) a promoter; (ii) a sequence encoding the immunogen, operably linked to the promoter; and optionally (iii) a selectable marker. Preferred vectors may further comprise (iv) an origin of replication; and (v) a transcription terminator downstream of and operably linked to (ii). In general, (i) & (v) will be eukaryotic and (iii) & (iv) will be prokaryotic.

Preferred promoters are viral promoters e.g. from cytomegalovirus (CMV). The vector may also include transcriptional regulatory sequences (e.g. enhancers) in addition to the promoter and which interact functionally with the promoter. Preferred vectors include the immediate-early CMV enhancer/promoter, and more preferred vectors also include CMV intron A. The promoter is operably linked to a downstream sequence encoding an immunogen, such that expression of the immunogen-encoding sequence is under the promoter's control.

Where a marker is used, it preferably functions in a microbial host (e.g. in a prokaryote, in a bacteria, in a yeast). The marker is preferably a prokaryotic selectable marker (e.g. transcribed under the control of a prokaryotic promoter). For convenience, typical markers are antibiotic resistance genes.

The vector is preferably an autonomously replicating episomal or extrachromosomal vector, such as a plasmid.

The vector preferably comprises an origin of replication. It is preferred that the origin of replication is active in prokaryotes but not in eukaryotes.

Preferred vectors thus include a prokaryotic marker for selection of the vector, a prokaryotic origin of replication, but a eukaryotic promoter for driving transcription of the immunogen-encoding sequence. The vectors will therefore (a) be amplified and selected in prokaryotic hosts without polypeptide expression, but (b) be expressed in eukaryotic hosts without being amplified. This arrangement is ideal for nucleic acid immunization vectors.

The vector may comprise a eukaryotic transcriptional terminator sequence downstream of the coding sequence. This can enhance transcription levels. Where the coding sequence does not have its own, the vector preferably comprises a polyadenylation sequence. A preferred polyadenylation sequence is from bovine growth hormone.

The vector may comprise a multiple cloning site

In addition to sequences encoding the immunogen and a marker, the vector may comprise a second eukaryotic coding sequence. The vector may also comprise an IRES upstream of said second sequence in order to permit translation of a second eukaryotic polypeptide from the same transcript as the immunogen. Alternatively, the immunogen-coding sequence may be downstream of an IRES.

The vector may comprise unmethylated CpG motifs e.g. unmethylated DNA sequences which have in common a cytosine preceding a guanosine, flanked by two 5′ purines and two 3′ pyrimidines. In their unmethylated form these DNA motifs have been demonstrated to be potent stimulators of several types of immune cell.

Vectors may be delivered in a targeted way. Receptor-mediated DNA delivery techniques are described in, for example, references 100 to 105. Therapeutic compositions containing a nucleic acid are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g. for enhancing or inhibiting levels of the encoded gene product) and efficacy of transformation and expression are considerations which will affect the dosage required for ultimate efficacy. Where greater expression is desired over a larger area of tissue, larger amounts of vector or the same amounts re-administered in a successive protocol of administrations, or several administrations to different adjacent or close tissue portions may be required to effect a positive therapeutic outcome. In all cases, routine experimentation in clinical trials will determine specific ranges for optimal therapeutic effect.

Vectors can be delivered using gene delivery vehicles. The gene delivery vehicle can be of viral or non-viral origin (see generally references 106 to 109).

Viral-based vectors for delivery of a desired nucleic acid and expression in a desired cell are well known in the art. Exemplary viral-based vehicles include, but are not limited to, recombinant retroviruses (e.g. references 110 to 120), alphavirus-based vectors (e.g. Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532); hybrids or chimeras of these viruses may also be used), poxvirus vectors (e.g. vaccinia, fowlpox, canarypox, modified vaccinia Ankara, etc.), adenovirus vectors, and adeno-associated virus (AAV) vectors (e.g. see refs. 121 to 126). Administration of DNA linked to killed adenovirus [127] can also be employed.

Non-viral delivery vehicles and methods can also be employed, including, but not limited to, polycationic condensed DNA linked or unlinked to killed adenovirus alone [e.g. 127], ligand-linked DNA [128], eukaryotic cell delivery vehicles cells [e.g. refs. 129 to 133] and nucleic charge neutralization or fusion with cell membranes. Naked DNA can also be employed. Exemplary naked DNA introduction methods are described in refs. 134 and 135. Liposomes (e.g. immunoliposomes) that can act as gene delivery vehicles are described in refs. 136 to 140. Additional approaches are described in references 141 & 142.

Further non-viral delivery suitable for use includes mechanical delivery systems such as the approach described in ref. 142. Moreover, the coding sequence and the product of expression of such can be delivered through deposition of photopolymerized hydrogel materials or use of ionizing radiation [e.g. refs. 143 & 144]. Other conventional methods for gene delivery that can be used for delivery of the coding sequence include, for example, use of hand-held gene transfer particle gun [145] or use of ionizing radiation for activating transferred genes [143 & 144].

Delivery of DNA using PLG {poly(lactide-co-glycolide)} microparticles is a particularly preferred method e.g. by adsorption to the microparticles, which are optionally treated to have a negatively-charged surface (e.g. treated with SDS) or a positively-charged surface (e.g. treated with a cationic detergent, such as CTAB).

### Methods of Treatment, and Administration of the Vaccine

The invention also provides a method for raising an immune response in a mammal comprising the step of administering an effective amount of a polypeptide, hybrid polypeptide, nucleic acid or an immunogenic composition as described above. The immune response is preferably protective and preferably involves antibodies and/or cell-mediated immunity. The method may raise a booster response.

The invention also provides a polypeptide, hybrid polypeptide, nucleic acid or an immunogenic composition described above for use as a medicament e.g. for use in raising an immune response in a mammal.

The invention also provides the use of a polypeptide, hybrid polypeptide, nucleic acid or an immunogenic composition described above in the manufacture of a medicament for raising an immune response in a mammal.

By raising an immune response in the mammal by these uses and methods, the mammal can be protected against disease and/or infection caused by GBS e.g. against meningitis.

The invention also provides a delivery device pre-filled with an immunogenic composition of the invention.

The mammal is preferably a human. The human may be a teenager or an adult.

One way of checking efficacy of therapeutic treatment involves monitoring GBS infection after administration of the compositions of the invention. One way of checking efficacy of prophylactic treatment involves testing post-immunisation sera in standard tests; for example, sera can be tested in an opsonophagocytic killing assay (OPKA), with the ability to opsonise bacteria indicating protective efficacy. Another way of checking efficacy of prophylactic treatment involves post-immunisation challenge in an animal model of GBS infection, e.g., guinea pigs or mice. One such model is described in reference 146. Another way of assessing the immunogenicity of the compositions of the present invention is to express the polypeptides recombinantly for screening patient sera or mucosal secretions by immunoblot and/or microarrays. A positive reaction between the polypeptide and the patient sample indicates that the patient has mounted an immune response to the polypeptide in question. This method may also be used to identify immunodominant antigens and/or epitopes within antigens.

Compositions of the invention will generally be administered directly to a patient. Direct delivery may be accomplished by parenteral injection (e.g. subcutaneously, intraperitoneally, intravenously, intramuscularly, or to the interstitial space of a tissue), or mucosally, such as by rectal, oral (e.g. tablet, spray), vaginal, topical, transdermal or transcutaneous, intranasal, ocular, aural, pulmonary or other mucosal administration.

The invention may be used to elicit systemic and/or mucosal immunity, preferably to elicit an enhanced systemic and/or mucosal immunity.

Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Vaccines prepared according to the invention may be used to treat both children and adults. Thus a human patient may be less than 1 year old, less than 5 years old, 1-5 years old, 5-15 years old, 15-55 years old, or at least 55 years old. Preferred patients for receiving the vaccines are adolescents (e.g. 13-20 years old), pregnant women, and the elderly (e.g. ≧50 years old, ≧60 years old, and preferably ≧65 years. The vaccines are not suitable solely for these groups, however, and may be used more generally in a population.

Vaccines produced by the invention may be administered to patients at substantially the same time as (e.g. during the same medical consultation or visit to a healthcare professional or vaccination centre) other vaccines e.g. at substantially the same time as a rubella vaccine, a varicella vaccine, a diphtheria vaccine, a tetanus vaccine, a pertussis vaccine, a DTP vaccine, an inactivated poliovirus vaccine, a hepatitis B virus vaccine, a meningococcal conjugate vaccine (such as a tetravalent A-C-W135-Y vaccine), a respiratory syncytial virus vaccine, an human papillomavirus vaccine, an influenza virus vaccines (including a pandemic influenza virus vaccine) etc.

Vaccines of the invention may also be administered to patients at substantially the same time as (e.g. during the same medical consultation or visit to a healthcare professional) an antiviral compound, and in particular an antiviral compound active against influenza virus (e.g. oseltamivir and/or zanamivir). These antivirals include neuraminidase inhibitors, such as a (3R,4R,5S)-4-acetylamino-5-amino-3(1-ethylpropoxy)-1-cyclohexene-1-carboxylic acid or 5-(acetylamino)-4-[(aminoiminomethyl)-amino]-2,6-anhydro-3,4,5-trideoxy-D-glycero-D-galactonon-2-enonic acid, including esters thereof (e.g. the ethyl esters) and salts thereof (e.g. the phosphate salts). A preferred antiviral is (3R,4R,5 S)-4-acetylamino-5-amino-3 (1-ethylpropoxy)-1-cyclohexene-1-carboxylic acid, ethyl ester, phosphate (1:1), also known as oseltamivir phosphate (TAMIFLU™).

### Combinations

In addition to a GBS67 polypeptide fragment, a composition may include: (i) one or more further polypeptides that elicit antibody responses against GBS proteins, particularly against GBS proteins other than GBS67; (ii) a capsular saccharide from GBS; and/or (iii) one or more further immunogens that elicit antibody responses that recognise epitopes on non-GBS organisms.

Combinations with Further Polypeptide Antigens

GBS67 polypeptide fragments described above may be combined with one or more (i.e. 1, 2, 3, 4, 5, 6, 7, 8, 9, or all 10) polypeptide antigens selected from the group consisting of: (1) a (GBS80) antigen; (2) a GBS59 antigen; (3) a GBS1523 antigen; (4) a GBS 104 antigen; (5) a GBS1524 antigen; (6) a GBS3 antigen; (7) a SAN1485 antigen; (8) a GBS147 antigen; (9) a GBS328 antigen; and/or (10) a GBS84 antigen.

These further antigens may be added as separate polypeptides. As an alternative, they may be added as hybrids e.g. a GBS80-GBS1523 hybrid. As a further alternative, they may be fused to a GBS67 polypeptide fragment to provide a hybrid polypeptide.

Any of these combinations may also include one or more GBS capsular saccharide(s), which will typically be conjugated to carrier protein(s). Further information about such saccharides and conjugation is provided below.

### GBS80

The original ‘GBS80’ (SAG0645) sequence was annotated in reference 147 as a cell wall surface anchor family protein (see GI: 22533660). For reference purposes, the amino acid sequence of full length GBS80 as found in the 2603 strain is given as SEQ ID NO: 34 herein. Preferred GBS80 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:34; and/or (b) comprising a fragment of at least ‘n’ consecutive amino acids of SEQ ID NO: 34, wherein ‘n’ is 7 or more (e.g. 8, 10, 12, 14, 16, 18, 20, 25, 30, 35, 40, 50, 60, 70, 80, 90, 100, 150, 200, 250 or more). These GBS80 proteins include variants of SEQ ID NO: 34.

Preferred fragments of (b) comprise an epitope from SEQ ID NO: 34. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 34 while retaining at least one epitope of SEQ ID NO: 34. Other fragments omit one or more protein domains.

Wild-type GBS-80 contains a N-terminal leader or signal sequence region at amino acids 1-37 of SEQ ID NO:34. One or more amino acids from the leader or signal sequence region of GBS80 can be removed, e.g. SEQ ID NO:35. The wild-type sequence also contains a C-terminal transmembrane region at amino acids 526-543 of SEQ ID NO: 34. One or more amino acids from the transmembrane region and/or a cytoplasmic region may be removed, e.g. SEQ ID NO:36. Wild-type GBS80 contains an amino acid motif indicative of a cell wall anchor at amino acids 521-525 of SEQ ID NO:34. In some recombinant host cell systems it may be useful to remove this motif to facilitate secretion of a recombinant GBS80 polypeptide from the host cell. Thus the transmembrane and/or cytoplasmic regions and the cell wall anchor motif may be removed from GBS80, e.g. SEQ ID NO:37. Alternatively, in some recombinant host cell systems it may be useful to use the cell wall anchor motif to anchor the recombinantly expressed polypeptide to the cell wall. The extracellular domain of the expressed polypeptide may be cleaved during purification or the recombinant polypeptide may be left attached to either inactivated host cells or cell membranes in the final composition, e.g. SEQ ID NO:38. A particularly immunogenic fragment of wild-type GBS80 is located towards the N-terminus of the polypeptide, and is SEQ ID NO:39.

### GBS59

GBS59 is the pilus backbone protein encoded by pathogenicity island 2a (BP-2a). For reference purposes, the amino acid sequence of full length GBS59 as found in the 2603 strain is given as SEQ ID NO: 40 herein. Preferred GBS59 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 40; and/or (b) comprising a fragment of at least ‘n’ consecutive amino acids of SEQ ID NO: 40, wherein ‘n’ is 7 or more (e.g. 8, 10, 12, 14, 16, 18, 20, 25, 30, 35, 40, 50, 60, 70, 80, 90, 100, 150, 200, 250 or more). These GBS59 proteins include variants of SEQ ID NO: 40. Preferred fragments of (b) comprise an epitope from SEQ ID NO: 40. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 40 while retaining at least one epitope of SEQ ID NO: 40. Other fragments omit one or more protein domains.

Variants of GBS59 exist in strains H36B, 515, CJB111, DK21 and CJB110. For reference purposes, the amino acid sequence of full length GBS59 as found in the H36B, 515, CJB111. CJB110 and DK21 strains are given as SEQ ID NOs: 41, 42, 43, 44, and 45. Preferred GBS59 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NOs: 41, 42, 43, 44, or 45; and/or (b) comprising a fragment of at least ‘n’ consecutive amino acids of SEQ ID NOs: 41, 42, 43, 44, or 45, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NOs: 41, 42, 43, 44, or 45. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NOs: 41, 42, 43, 44, or 45 while retaining at least one epitope of SEQ ID NOs: 41, 42, 43, 44, or 45. Other fragments omit one or more protein domains.

### GBS1523

The original ‘GBS1523’ (SAN1518; SpbI) sequence was annotated in reference 3 as a cell wall surface anchor family protein (see GI: 77408651). For reference purposes, the amino acid sequence of full length GBS1523 as found in the COH1 strain is given as SEQ ID NO: 46 herein. Preferred GBS 1523 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:46; and/or (b) comprising a fragment of at least ‘n’ consecutive amino acids of SEQ ID NO: 46, wherein ‘n’ is 7 or more (e.g. 8, 10, 12, 14, 16, 18, 20, 25, 30, 35, 40, 50, 60, 70, 80, 90, 100, 150, 200, 250 or more). These GBS1523 proteins include variants of SEQ ID NO: 46. Preferred fragments of (b) comprise an epitope from SEQ ID NO: 46. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 46 while retaining at least one epitope of SEQ ID NO: 46. Other fragments omit one or more protein domains.

Wild-type GBS 1523 contains a N-terminal leader or signal sequence region at amino acids 1 to 29 of SEQ ID NO:46 which may be removed in fragments, e.g. SEQ ID NO:47. The wild-type sequence contains an amino acid motif indicative of a cell wall anchor (LPSTG) at amino acids 468-472 of SEQ ID NO:46. In some recombinant host cell systems, it may be preferable to remove this motif to facilitate secretion of a recombinant polypeptide from the cell. Alternatively, it may be preferable to use the cell wall anchor motif to anchor the recombinantly expressed polypeptide to the cell wall. The extracellular domain of the expressed polypeptide may be cleaved during purification or the recombinant polypeptide may be left attached to either inactivated host cells or cell membranes in the final composition. An E box containing a conserved glutamic residue has also been identified at amino acids 419-429 of SEQ ID NO:46, with a conserved glutamic acid at residue 423. The E box motif may be important for the formation of oligomeric pilus-like structures, and so useful fragments of GBS 1523 may include the conserved glutamic acid residue. A mutant of GBS1523 has been identified in which the glutamine (Q) at position 41 of SEQ ID NO:46 is substituted for a lysine (K), as a result of a mutation of a codon in the encoding nucleotide sequence from CAA to AAA. This substitution may be present in the GBS1523 sequences and GBS1523 fragments (e.g. SEQ ID NO:48).

Where the compositions include both GBS80 and GBS1523, a hybrid polypeptide may be used. Examples of GBS80-GBS1523 hybrids are found in reference 148 and include the polypeptides of SEQ ID NOS: 49-52.

### GBS104

The original ‘GBS104’ (SAG0649) sequence was annotated in reference 147 as ‘a cell wall surface anchor family protein’ (see GI: 22533664). For reference purposes, the amino acid sequence of full length GBS104 as found in the 2603 strain is given as SEQ ID NO: 53 herein. Preferred GBS 104 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 53; and/or (b) comprising a fragment of at least ‘n’ consecutive amino acids of SEQ ID NO: 53, wherein ‘n’ is 7 or more (e.g. 8, 10, 12, 14, 16, 18, 20, 25, 30, 35, 40, 50, 60, 70, 80, 90, 100, 150, 200, 250 or more). These GBS 104 proteins include variants of SEQ ID NO: 53. Preferred fragments of (b) comprise an epitope from SEQ ID NO: 40. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 53 while retaining at least one epitope of SEQ ID NO: 53. Other fragments omit one or more protein domains.

### GBS 1524

For reference purposes, the amino acid sequence of full length GBS1524 (SAN1519) as found in the COH1 strain is given as SEQ ID NO: 54 herein. Preferred GBS1524 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 54; and/or (b) comprising a fragment of at least ‘n’ consecutive amino acids of SEQ ID NO: 54, wherein ‘n’ is 7 or more (e.g. 8, 10, 12, 14, 16, 18, 20, 25, 30, 35, 40, 50, 60, 70, 80, 90, 100, 150, 200, 250 or more). These GBS1524 proteins include variants of SEQ ID NO: 54. Preferred fragments of (b) comprise an epitope from SEQ ID NO: 54. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 54 while retaining at least one epitope of SEQ ID NO: 54. Other fragments omit one or more protein domains.

### GBS3

The original ‘GBS3’ (SAG2603; BibA) sequence was annotated in reference 147 as ‘a pathogenicity protein’ (see GI:22535109). For reference purposes, the amino acid sequence of full length GBS3 as found in the 2603 strain is given as SEQ ID NO: 55 herein. Preferred GBS3 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 55; and/or (b) comprising a fragment of at least ‘n’ consecutive amino acids of SEQ ID NO: 55, wherein ‘n’ is 7 or more (e.g. 8, 10, 12, 14, 16, 18, 20, 25, 30, 35, 40, 50, 60, 70, 80, 90, 100, 150, 200, 250 or more). These GBS3 proteins include variants of SEQ ID NO: 55. Preferred fragments of (b) comprise an epitope from SEQ ID NO: 35. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 55 while retaining at least one epitope of SEQ ID NO: 55. Other fragments omit one or more protein domains.

Wild-type GBS3 contains a N-terminal leader or signal sequence region at amino acids 1 to 36 of SEQ ID NO:55 which may be removed in fragments, e.g. SEQ ID NO563. GBS3 also contains an amino acid motif indicative of a cell wall anchor (LPXTG), a transmembrane region and cytoplasmic domains (see reference 149). The leader or signal sequence region, the transmembrane and cytoplasmic domains, and the cell wall anchor motif may all be removed from GBS3 to leave a fragment comprising the coiled-coil and proline-rich segments as set forth below (SEQ ID NO:57). Alternative fragments of GBS3 may comprise: the signal sequence region and coiled coil segment (SEQ ID NO:58); the coiled coil segment (SEQ ID NO:59); or the signal sequence region, coiled coil segment, and proline-rich segment (SEQ ID NO:60).

Variants of GBS3 exist in the 515 strain (SAL2118), CJB111 strain (SAM1974) and COH1 strain (SAN2207). Reference amino acid sequences for full-length GBS3 in the 515 strain, the CJB111 strain and the COH1 strain are given herein as SEQ ID NO: 61, SEQ ID NO:62 and SEQ ID NO:63 respectively. Thus, GBS3 polypeptides for use with the invention may also comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 61, SEQ ID NO:62 or SEQ ID NO:63; and/or (b) comprising a fragment of at least ‘n’ consecutive amino acids of SEQ ID NO: 61, SEQ ID NO:62 or SEQ ID NO:63, wherein ‘n’ is 7 or more (e.g. 8, 10, 12, 14, 16, 18, 20, 25, 30, 35, 40, 50, 60, 70, 80, 90, 100, 150, 200, 250 or more). These GBS3 proteins include variants of SEQ ID NO: 61, SEQ ID NO:62 or SEQ ID NO:63. Preferred fragments of (b) comprise an epitope from SEQ ID NO: 61, SEQ ID NO:62 or SEQ ID NO:63. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 61, SEQ ID NO:62 or SEQ ID NO:63 while retaining at least one epitope of SEQ ID NO: 61, SEQ ID NO:62 or SEQ ID NO:63. Other fragments omit one or more protein domains.

The invention includes the use of fragments of GBS3 from the 515, cjb111 and coh1 strains that are analogous to fragments of GBS3 from the 2603 strain discussed in detail above, e.g. lacking the N-terminal leader or signal sequence region; comprising the coiled-coil and proline-rich segments; comprising the signal sequence region and coiled coil segment; comprising the coiled coil segment; or comprising the signal sequence region, coiled coil segment, and proline-rich segment.

### SAN1485

The original ‘SAN1485’ sequence was annotated in reference 3 as ‘cell wall surface anchor family protein’ (see GI: 77408233). For reference purposes, the amino acid sequence of full length SAN1485 as found in the COH1 strain is given as SEQ ID NO: 64 herein. Preferred SAN1485 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 64; and/or (b) comprising a fragment of at least ‘n’ consecutive amino acids of SEQ ID NO: 64, wherein ‘n’ is 7 or more (e.g. 8, 10, 12, 14, 16, 18, 20, 25, 30, 35, 40, 50, 60, 70, 80, 90, 100, 150, 200, 250 or more). These SAN1485 proteins include variants of SEQ ID NO: 64. Preferred fragments of (b) comprise an epitope from SEQ ID NO: 64. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 64 while retaining at least one epitope of SEQ ID NO: 64. Other fragments omit one or more protein domains.

### GBS 147

The original ‘GBS 147’ (SAG0416) sequence was annotated in reference 147 as ‘a putative protease’ (see GI: GI:22533435). For reference purposes, the amino acid sequence of full length GBS147 as found in the 2603 strain is given as SEQ ID NO: 65 herein. Preferred GBS 147 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 65 and/or (b) comprising a fragment of at least ‘n’ consecutive amino acids of SEQ ID NO: 65, wherein ‘n’ is 7 or more (e.g. 8, 10, 12, 14, 16, 18, 20, 25, 30, 35, 40, 50, 60, 70, 80, 90, 100, 150, 200, 250 or more). These GBS147 proteins include variants of SEQ ID NO: 65. Preferred fragments of (b) comprise an epitope from SEQ ID NO: 65. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 65 while retaining at least one epitope of SEQ ID NO: 65.

### GBS328

The original ‘GBS328’ (SAG1333) sequence was annotated in reference 147 as ‘5′-nucleotidase family protein’ (see GI: 22534359). For reference purposes, the amino acid sequence of full length GBS328 as found in the 2603 strain is given as SEQ ID NO: 66 herein. Preferred GBS328 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 66; and/or (b) comprising a fragment of at least ‘n’ consecutive amino acids of SEQ ID NO: 66, wherein ‘n’ is 7 or more (e.g. 8, 10, 12, 14, 16, 18, 20, 25, 30, 35, 40, 50, 60, 70, 80, 90, 100, 150, 200, 250 or more). These GBS328 proteins include variants of SEQ ID NO: 66. Preferred fragments of (b) comprise an epitope from SEQ ID NO: 66. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 66 while retaining at least one epitope of SEQ ID NO: 66. Other fragments omit one or more protein domains.

### GBS84

The original ‘GBS84’ (SAG0907) sequence was annotated in reference 147 as ‘a putative lipoprotein’ (see GI: 22533929). For reference purposes, the amino acid sequence of full length GBS84 as found in the 2603 strain is given as SEQ ID NO: 67 herein. Preferred GBS84 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 67; and/or (b) comprising a fragment of at least ‘n’ consecutive amino acids of SEQ ID NO: 67, wherein ‘n’ is 7 or more (e.g. 8, 10, 12, 14, 16, 18, 20, 25, 30, 35, 40, 50, 60, 70, 80, 90, 100, 150, 200, 250 or more). These GBS84 proteins include variants of SEQ ID NO: 67. Preferred fragments of (b) comprise an epitope from SEQ ID NO: 67. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 67 while retaining at least one epitope of SEQ ID NO: 67. Other fragments omit one or more protein domains.

Combinations with GBS Saccharides

GBS67 polypeptide fragments may be combined with one or more GBS capsular saccharide(s), which will typically be conjugated to carrier protein(s). Thus the invention provides an immunogenic composition comprising a combination of:
(1) a GBS67 polypeptide fragment as discussed above; and
(2) one or more GBS capsular saccharides.

A saccharide used in component (2) of this combination is ideally present as a conjugate comprising a saccharide moiety and a carrier protein moiety. The carrier moiety in the conjugate may be a single GBS67 polypeptide fragment, a hybrid GBS67 polypeptide, a non-GBS67 GBS polypeptide, or a non-GBS polypeptide.

The saccharide is from the capsular saccharide of GBS. The saccharide may be a polysaccharide having the size that arises during purification of the saccharide from bacteria, or it may be an oligosaccharide achieved by fragmentation of such a polysaccharide.

A composition may include a capsular saccharide from one or more of the following streptococcal serotypes: Ia, Ib, Ia/c, II, III, IV, V, VI, VII and VIII. A composition may include multiple serotypes e.g. 2, 3, 4, 5, 6, 7, or 8 serotypes. Including a saccharide from one or more of serotypes Ia, Ib, II, III & V is useful. The capsular saccharides of each of these five serotypes include: (a) a terminal N-acetyl-neuraminic acid (NeuNAc) residue (commonly referred to as sialic acid), which in all cases is linked 2→3 to a galactose residue; and (b) a N-acetyl-glucosamine residue (GlcNAc) within the trisaccharide core.

Saccharides used according to the invention may be in their native form, or may have been modified. For example, the saccharide may be shorter than the native capsular saccharide, or may be chemically modified. For instance, the saccharide may be de-O-acetylated (partially or fully), de-N-acetylated (partially or fully) or N-propionated (partially or fully), etc. De-acetylation may occur before, during or after conjugation, but preferably occurs before conjugation. Depending on the particular saccharide, de-acetylation may or may not affect immunogenicity. The relevance of O-acetylation on GBS saccharides in various serotypes is discussed in ref. 150, and in some embodiments O-acetylation of sialic acid residues at positions 7, 8 and/or 9 is retained before, during and after conjugation e.g. by protection/de-protection, by re-acetylation, etc. However, typically the GBS saccharide used in the present invention has substantially no O-acetylation of sialic acid residues at positions 7, 8 and/or 9. The effect of de-acetylation etc. can be assessed by routine assays. Another possible modification is the removal of sialic acid residues from the saccharide, such as side-chain terminal sialic acids [151]. In particular, when a serotype V capsular saccharide is used in the invention, it may be modified by desialylation as described in ref. [151]. Desialylated GBS serotype V capsular saccharide may be prepared by treating purified GBS serotype V capsular saccharide under mildly acidic conditions (e.g. 0.1M sulphuric acid at 80° C. for 60 minutes) or by treatment with neuraminidase, as described in ref. [151]. In another example, full-length polysaccharides may be depolymerised to give shorter fragments for use with the invention e.g. by hydrolysis in mild acid, by heating, by sizing chromatography, etc. Chain length has been reported to affect immunogenicity of GBS saccharides in rabbits [152]. In particular, when a serotype II and/or III capsular saccharide is used in the invention, it may be depolymerised as described in ref. 153. This document describes the partial depolymerization of type II and type III capsular saccharides by mild deaminative cleavage to antigenic fragments with reducing-terminal 2,5-anhydro-D-mannose residues.

Capsular saccharides can be purified by known techniques, as described in the references herein such as ref. 154. A typical process involves base extraction, centrifugation, filtration, RNase/DNase treatment, protease treatment, concentration, size exclusion chromatography, ultrafiltration, anion exchange chromatography, and further ultrafiltration. As an alternative, the purification process described in ref. 155 can be used. This process involves base extraction, ethanol/CaCl₂ treatment, CTAB precipitation, and re-solubilisation.

The invention is not limited to saccharides purified from natural sources, however, and the saccharides may be obtained by other methods, such as total or partial synthesis. Saccharides will typically be conjugated to a carrier protein. In general, covalent conjugation of saccharides to carriers enhances the immunogenicity of saccharides as it converts them from T-independent antigens to T-dependent antigens, thus allowing priming for immunological memory.

Conjugation of GBS saccharides has been widely reported e.g. see refs. 156 to 163. The typical prior art process for GBS saccharide conjugation involves reductive amination of a purified saccharide to a carrier protein such as tetanus toxoid (TT) or CRM197 [157]. The reductive amination involves an amine group on the side chain of an amino acid in the carrier and an aldehyde group in the saccharide. As GBS capsular saccharides do not include an aldehyde group in their natural form then this is typically generated before conjugation by oxidation (e.g. periodate oxidation) of a portion of the saccharide's sialic acid residues [157, 164]. Conjugate vaccines prepared in this manner have been shown to be safe and immunogenic in humans for each of GBS serotypes Ia, Ib, II, III, and V [165].

Preferred carrier proteins are bacterial toxins, such as diphtheria or tetanus toxins, or toxoids or mutants thereof. These are commonly used in conjugate vaccines. A carrier protein in a conjugate may or may not be one of the GBS59 antigens of (1). If it is not a GBS59 antigen it may instead be a different GBS antigen. In some embodiments, though, the carrier is not a GBS antigen, and may be e.g. a bacterial toxin or toxoid.

Typical carrier proteins are diphtheria or tetanus toxoids or mutants thereof. Fragments of toxins or toxoids can also be used e.g. fragment C of tetanus toxoid [166]. The CRM197 mutant of diphtheria toxin [167-169] is a particularly useful with the invention. Other suitable carrier proteins include N. meningitidis outer membrane protein complex [170], synthetic peptides [171,172], heat shock proteins [173,174], pertussis proteins [175,176], cytokines [177], lymphokines [187], hormones [187], growth factors, artificial proteins comprising multiple human CD4⁺ T cell epitopes from various pathogen-derived antigens [178] such as N19 [179], protein D from H. influenzae [180-182], iron-uptake proteins [183], toxin A or B from C. difficile [184], recombinant P. aeruginosa exoprotein A (rEPA) [185], etc.

Where a composition includes more than one conjugate, each conjugate may use the same carrier protein or a different carrier protein.

In some embodiments, a single conjugate may carry saccharides from multiple serotypes [186]. Usually, however, each conjugate will include saccharide from a single serotype.

Conjugates may have excess carrier (w/w) or excess saccharide (w/w). In some embodiments, a conjugate may include equal weights of each. For example, conjugates with a saccharide:protein ratio (w/w) of between 1:5 and 5:1 may be used, in particular ratios between 1:5 and 2:1.

The carrier molecule may be covalently conjugated to the carrier directly or via a linker. Direct linkages to the protein may be achieved by, for instance, reductive amination between the saccharide and the carrier, as described in, for example, references 187 and 188. The saccharide may first need to be activated e.g. by oxidation. Linkages via a linker group may be made using any known procedure, for example, the procedures described in references 189 and 190. A preferred type of linkage is an adipic acid linker, which may be formed by coupling a free —NH₂ group (e.g. introduced to a glucan by amination) with adipic acid (using, for example, diimide activation), and then coupling a protein to the resulting saccharide-adipic acid intermediate [191,192]. Another preferred type of linkage is a carbonyl linker, which may be formed by reaction of a free hydroxyl group of a saccharide CDI [193, 194] followed by reaction with a protein to form a carbamate linkage. Other linkers include β-propionamido [195], nitrophenyl-ethylamine [196], haloacyl halides [197], glycosidic linkages [198], 6-aminocaproic acid [199], ADH [200], C₄ to C₁₂ moieties [201], etc. Carbodiimide condensation can also be used [202].

Combinations with Non-GBS Antigens

The GBS67 fragments may be used in combination with non-GBS antigens. Thus the invention provides an immunogenic composition comprising a combination of:
(1) a GBS67 polypeptide fragment as discussed above; and
(2) one or more antigen(s) selected from the group consisting of: diphtheria toxoid; tetanus toxoid; one or more pertussis antigens; hepatitis B virus surface antigen; an inactivated poliovirus antigen; a conjugate of the capsular saccharide antigen from serogroup C of Neisseria meningitidis; a conjugate of the capsular saccharide antigen from serogroup Y of Neisseria meningitidis; a conjugate of the capsular saccharide antigen from serogroup W135 of Neisseria meningitidis; a conjugate of the capsular saccharide antigen from serogroup A of Neisseria meningitides; one or more influenza antigens; and one or more human papillomavirus antigens.

Diphtheria toxoid can be obtained by treating (e.g. using formaldehyde) diphtheria toxin from Corynebacterium diphtherias. Diphtheria toxoids are disclosed in more detail in, for example, chapter 13 of reference 203.

Tetanus toxoid can be obtained by treating (e.g. using formaldehyde) tetanus toxin from Clostridium tetani. Tetanus toxoids are disclosed in more detail in chapter 27 of reference 203.

Pertussis antigens in vaccines are either cellular (whole cell, Pw) or acellular (Pa). The invention can use either sort of pertussis antigen. Preparation of cellular pertussis antigens is well documented (e.g. see chapter 21 of reference 203) e.g. it may be obtained by heat inactivation of phase I culture of B. pertussis. Acellular pertussis antigen(s) comprise specific purified B. pertussis antigens, either purified from the native bacterium or purified after expression in a recombinant host. It is usual to use more than one acellular antigen, and so a composition may include one, two or three of the following well-known and well-characterized B. pertussis antigens: (1) detoxified pertussis toxin (pertussis toxoid, or ‘PT’); (2) filamentous hemagglutinin (‘FHA’); (3) pertactin (also known as the ‘69 kiloDalton outer membrane protein’). FHA and pertactin may be treated with formaldehyde prior to use according to the invention. PT may be detoxified by treatment with formaldehyde and/or glutaraldehyde but, as an alternative to this chemical detoxification procedure, it may be a mutant PT in which enzymatic activity has been reduced by mutagenesis [204]. Further acellular pertussis antigens that can be used include fimbriae (e.g. agglutinogens 2 and 3).

Hepatitis B virus surface antigen (HBsAg) is the major component of the capsid of hepatitis B virus. It is conveniently produced by recombinant expression in a yeast, such as a Saccharomyces cerevisiae.

Inactivated poliovirus (IPV) antigens are prepared from viruses grown on cell culture and then inactivated (e.g. using formaldehyde). Because poliomyelitis can be caused by one of three types of poliovirus, as explained in chapter 24 of reference 203, a composition may include three poliovirus antigens: poliovirus Type 1 (e.g. Mahoney strain), poliovirus Type 2 (e.g. MEF-1 strain), and poliovirus Type 3 (e.g. Saukett strain).

When a composition includes one of diphtheria toxoid, tetanus toxoid or an acellular pertussis antigen in component (2) then it will usually include all three of them i.e. component (2) will include a D-T-Pa combination.

When a composition includes one of diphtheria toxoid, tetanus toxoid or a cellular pertussis antigen in component (2) then it will usually include all three of them i.e. component (2) will include a D-T-Pw combination.

Human papillomavirus antigens include L1 capsid proteins, which can assemble to form structures known as virus-like particles (VLPs). The VLPs can be produced by recombinant expression of L1 in yeast cells (e.g. in S. cerevisiae) or in insect cells (e.g. in Spodoptera cells, such as S. frugiperda, or in Drosophila cells). For yeast cells, plasmid vectors can carry the L1 gene(s); for insect cells, baculovirus vectors can carry the L 1 gene(s). More preferably, the composition includes L1 VLPs from both HPV-16 and HPV-18 strains. This bivalent combination has been shown to be highly effective [205]. In addition to HPV-16 and HPV-18 strains, it is also possible to include L1 VLPs from HPV-6 and HPV-11 strains to give a tetravalent combination.

Influenza antigens may be in the form of currently an influenza virus vaccine. Various forms of influenza virus vaccine are currently available (e.g. see chapters 17 & 18 of reference [203]). Vaccines are generally based either on live virus, inactivated virus, recombinant hemagglutinin or virosomes. Inactivated vaccines may be based on whole virions, split virions, or on purified surface antigens. The antigen in vaccines of the invention may take the form of a live virus or, more preferably, an inactivated virus. The vaccine can be, for instance, a trivalent vaccine (e.g. including hemagglutinin from a A/H1N1 strain, a A/H3N2 strain and a B strain). In other embodiments the vaccine is a monovalent vaccine (e.g. including hemagglutinin from a A/H1N1 strain or a A/H5N1 strain). The vaccine can be adjuvanted (e.g. with an oil-in-water emulsion) or unadjuvanted.

Human papillomavirus antigens are in the form of hollow virus-like particles (VLPs) assembled from recombinant HPV coat proteins, typically from HPV types 16 and 18, and optionally also from HPV types 6 and 11.

### Antibodies

Antibodies against GBS antigens can be used for passive immunisation [206]. Thus the invention provides a combination of antibodies for simultaneous, separate or sequential administration, wherein the combination includes at least two of: (a) an antibody which recognises a first amino acid sequence as defined above; (b) an antibody which recognises a second amino acid sequence as defined above; and/or (c) an antibody which recognises a third amino acid sequence as defined above;

The invention also provides the use of such antibody combinations in therapy. The invention also provides the use of such antibody combinations in the manufacture of a medicament. The invention also provides a method for treating a mammal comprising the step of administering to the mammal an effective amount of such a combination. As described above for immunogenic compositions, these methods and uses allow a mammal to be protected against GBS infection.

The term “antibody” includes intact immunoglobulin molecules, as well as fragments thereof which are capable of binding an antigen. These include hybrid (chimeric) antibody molecules [207, 208]; F(ab′)2 and F(ab) fragments and Fv molecules; non-covalent heterodimers [209, 210]; single-chain Fv molecules (sFv) [211]; dimeric and trimeric antibody fragment constructs; minibodies [212, 213]; humanized antibody molecules [214-216]; and any functional fragments obtained from such molecules, as well as antibodies obtained through non-conventional processes such as phage display. Preferably, the antibodies are monoclonal antibodies. Methods of obtaining monoclonal antibodies are well known in the art. Humanised or fully-human antibodies are preferred.

### General

The practice of the present invention will employ, unless otherwise indicated, conventional methods of chemistry, biochemistry, molecular biology, immunology and pharmacology, within the skill of the art. Such techniques are explained fully in the literature. See, e.g., references 217-224, etc.

“GI” numbering is used above. A GI number, or “GenInfo Identifier”, is a series of digits assigned consecutively to each sequence record processed by NCBI when sequences are added to its databases. The GI number bears no resemblance to the accession number of the sequence record. When a sequence is updated (e.g. for correction, or to add more annotation or information) then it receives a new GI number. Thus the sequence associated with a given GI number is never changed.

Where the invention concerns an “epitope”, this epitope may be a B-cell epitope and/or a T-cell epitope. Such epitopes can be identified empirically (e.g. using PEPSCAN [225,226] or similar methods), or they can be predicted (e.g. using the Jameson-Wolf antigenic index [227], matrix-based approaches [228], MAPITOPE [229], TEPITOPE [230,231], neural networks [232], OptiMer & EpiMer [233, 234], ADEPT [235], Tsites [236], hydrophilicity [237], antigenic index [238] or the methods disclosed in references 239-243, etc.). Epitopes are the parts of an antigen that are recognised by and bind to the antigen binding sites of antibodies or T-cell receptors, and they may also be referred to as “antigenic determinants”.

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Antibodies will generally be specific for their target. Thus they will have a higher affinity for the target than for an irrelevant control protein, such as bovine serum albumin.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref. 244. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref 245.

### BRIEF DESCRIPTION OF THE DRAWINGS

FIG. 1: Alignment of GBS67 from 2603 (SAG1408) and H37B (SAI1512) showing location of fragment 1 (Fr1), fragment 2 (Fr2) and fragment 3 (Fr3).

FIG. 2: Purification of fragment 1 (Fr1), fragment 2 (Fr2) and fragment 3 (Fr3) from 2603 (FIG. 2A) and H36B (FIG. 2B)

FIG. 3: Polyclonal antibodies raised against GBS67 2603 (FIG. 3A) and GBS67 H36B (FIG. 3B) recognise fragment 3 (Fr 3) from both variants in a Western blot analysis but not fragment 2 (Fr 2) or fragment 1 (Fr 1).

FIG. 4: Antibodies against Fragment 1 from 2603 only recognize recombinant Fragment 1 from 2603 variant (1 2603) and full-length GBS67 from 2603 variant (FL 2603) in a Western blot analysis. Full-length GBS67 from H36B (FL H36B), fragments 1, 2 and 3 from H36B and fragments 2 and 3 from 2603 not recognized.

FIG. 5A: Antibodies against Fragment 2 from 2603 recognize recombinant Fragment 2 from 2603 variant (2 2603) and H36B variant (2 2603), as well as full-length GBS67 from 2603 variant (FL 2603) in a Western blot analysis.

FIG. 5B: Antibodies against Fragment 3 from 2603 recognize recombinant Fragment 3 from 2603 variant (3 2603) and H36B variant (3 2603), as well as full-length GBS67 from 2603 variant (FL 2603) and full-length GBS67 from H36B (FL H36B) in a Western blot analysis.

### MODES FOR CARRYING OUT THE INVENTION

### GBS67 Variants are Cross-Protective

Two allelic variants of GBS67 (AP1-2a) have been identified, one in GBS strain 2603 and one in GBS strain H36B. The GBS67 strain identified in GBS strain 2603 is predominant, being the variant that is present in 87% of GBS strains.

Either of these two GBS67 variants is capable of conferring cross-protection against GBS strains expressing the other GBS67 variant. For example, as shown in Table 1 below, the pups of female mice immunized with GBS67 (AP1-2a) from the 2603 strain are protected against challenge with GBS strains expressing either the 2603 or the H36B variant of GBS67.

TABLE 1
GBS67 confers cross-protection in GBS mouse maternal
immunization/pup challenge model

| | | | | Statistical |
|---|---|---|---|---|
| | GBS strain | | Protection | significance |
| Antigen | (serotype) | Allelic variant | % | p value |
| AP-2a | CJB111 (V) | CJB111 | 69.6 | <0.0001 |
| 2603 variant | 515 (Ia) | 515 | 61.9 | 0.0018 |
| | 3050 (II) | 2603 | 94.4 | <0.0001 |
| | 5401 (II) | H36B | 62.8 | <0.0001 |
| AP1 -2a | 515 (Ia) | 515 | 57.4 | <0.0001 |
| H36B variant | 5401 (II) | H36B | 58.7 | <0.0001 |
| | DK21 (II) | H36B | 60.2 | <0.0001 |

An investigation was conducted to identify the portions of the GBS67 variants that are responsible for cross-protection.

### Identification of Three Fragments of GBS67

No crystal structure is available for AP1-2a (GBS67). An in silico analysis of the secondary structure of the GBS67 variants from 2603 and H36B identified three putative conserved fragments that might be responsible for cross-protective activity of GBS67 (see Table 2).

### Table 2: Conserved Fragments of GBS67 Variants

| | Amino | SEQ | Number of | Theoretical |
|---|---|---|---|---|
| | acid | ID | amino acid | MW |
| Fragment | residues | NO | residues | (kDa) |
| Fragment 1 2603 | 24-217 | 3 | 194 | 23.5 |
| Fragment 2 2603 | 218-615 | 4 | 398 | 47.3 |
| Fragment 3 2603 | 616-866 | 5 | 251 | 30.4 |
| Fragment 1 H36B | 24-217 | 6 | 194 | 24 |
| Fragment 2 H36B | 218-610 | 7 | 393 | 46.8 |
| Fragment 3 H36B | 611-861 | 8 | 251 | 30.5 |

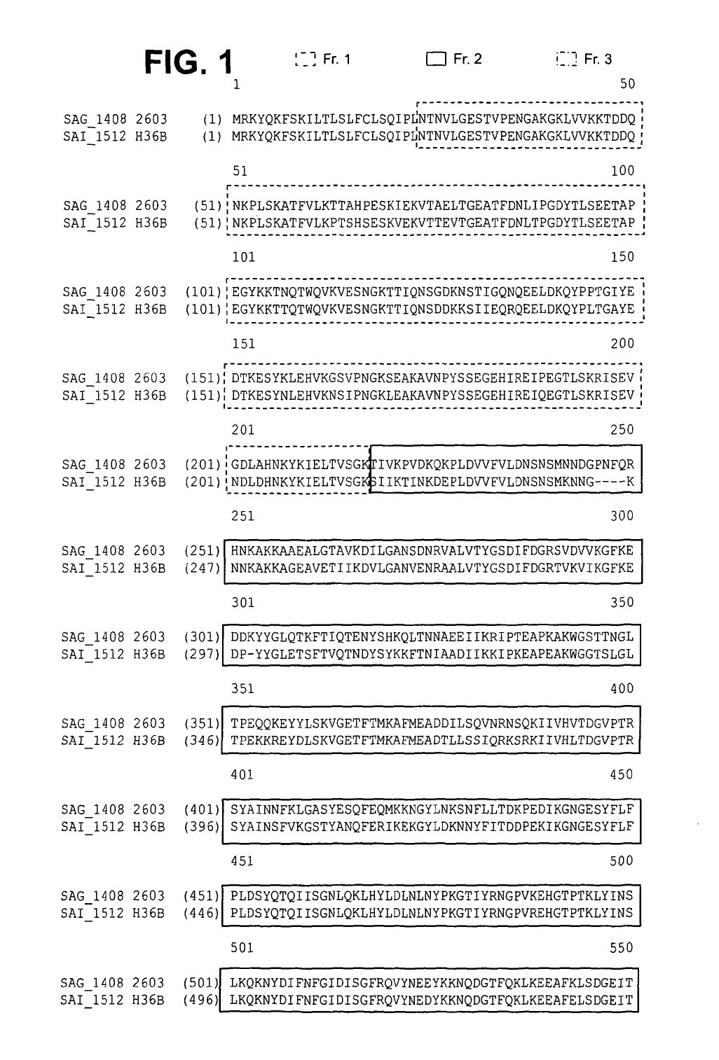

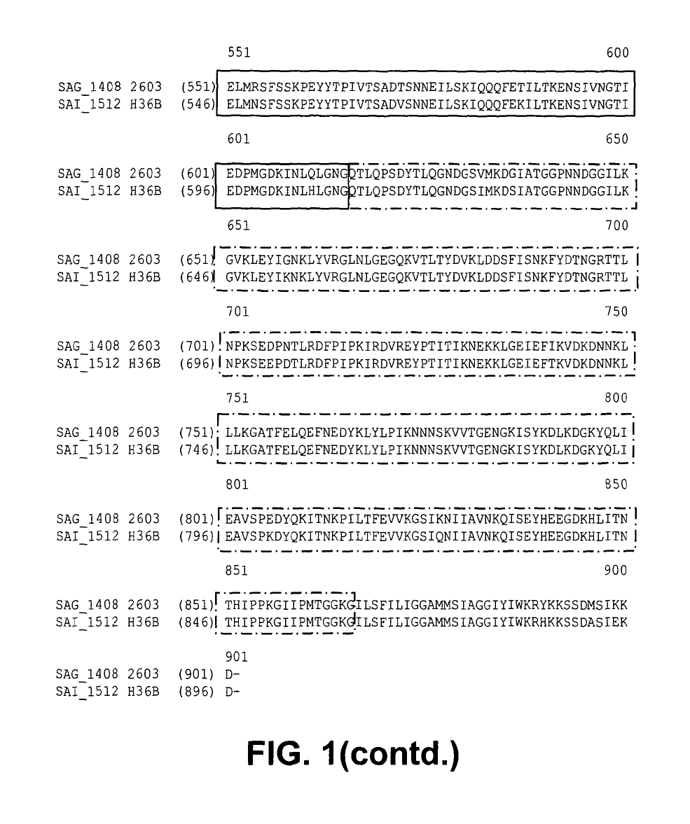

An alignment of the GBS67 variants showing the location of the 3 fragments is shown in FIG. 1.

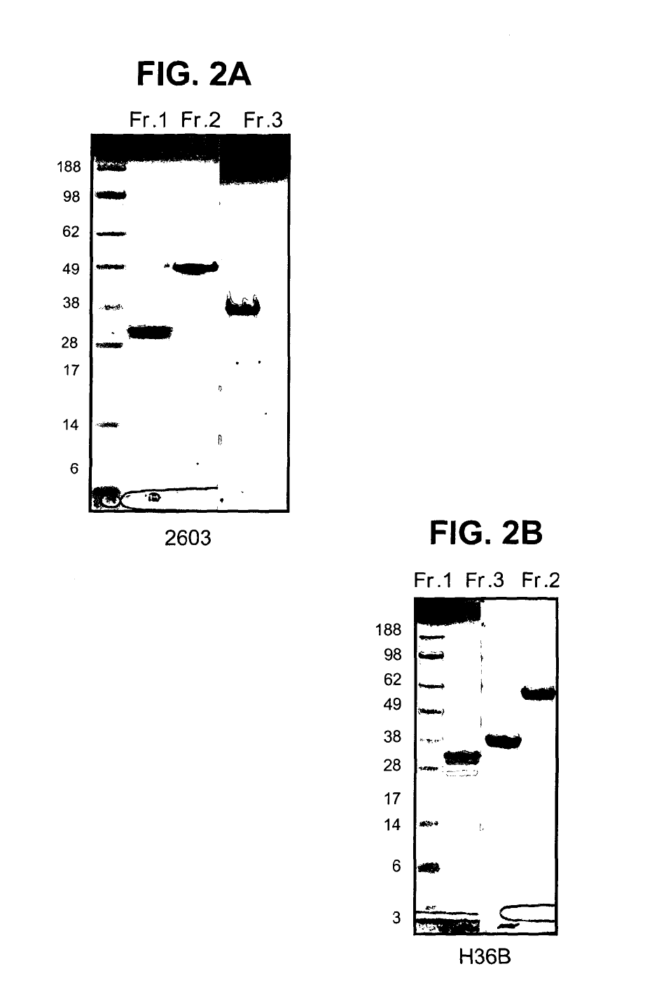

These six fragments were cloned and expressed as His-tagged proteins in HK100 and BL21(DE3) strains. All of the fragments were over-expressed and soluble. The fragments yields obtained are shown in Table 3 below and FIG. 2 shows the purified fragments isolated on gels.

TABLE 3

| | | | |
|---|---|---|---|
| Fragment | mg/ml | ml | mg total |

a: Purification of GBS67 2603 fragments

| | | | |
|---|---|---|---|
| GBS67-2603 1 His | 2.600 | 6.0 | 15.602 |
| GBS67-2603 2 His | 3.084 | 6.0 | 18.501 |
| GBS67-2603 3 His | 3.099 | 5.0 | 15.495 |

b: Purification of GBS67 H36B fragments

| | | | |
|---|---|---|---|
| GBS67-H36B 1 His | 3.157 | 5.5 | 17.364 |
| GBS67-H36B 2 His | 5.153 | 3.0 | 15.459 |
| GBS67-H36B 3 His | 9.470 | 3.0 | 28.410 |

### Assessment of Cross-Protective Activity of GBS67 Fragments

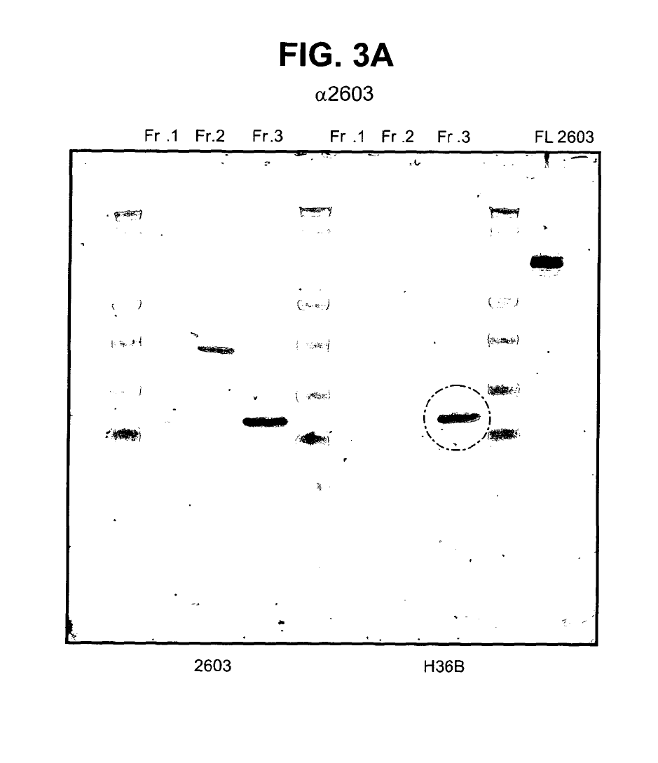

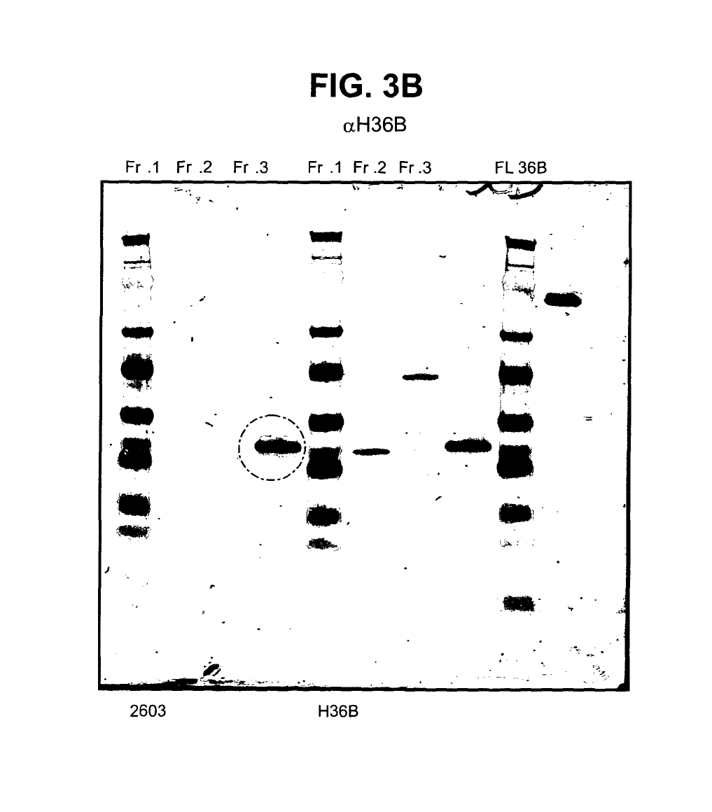

Polyclonal antibodies raised against the 2603 and H36B GBS67 variants were capable of recognizing fragment 3 from both variants in a Western Blot analysis (FIG. 3), suggesting that fragment 3 contains the epitopes responsible for inducing cross-protection.

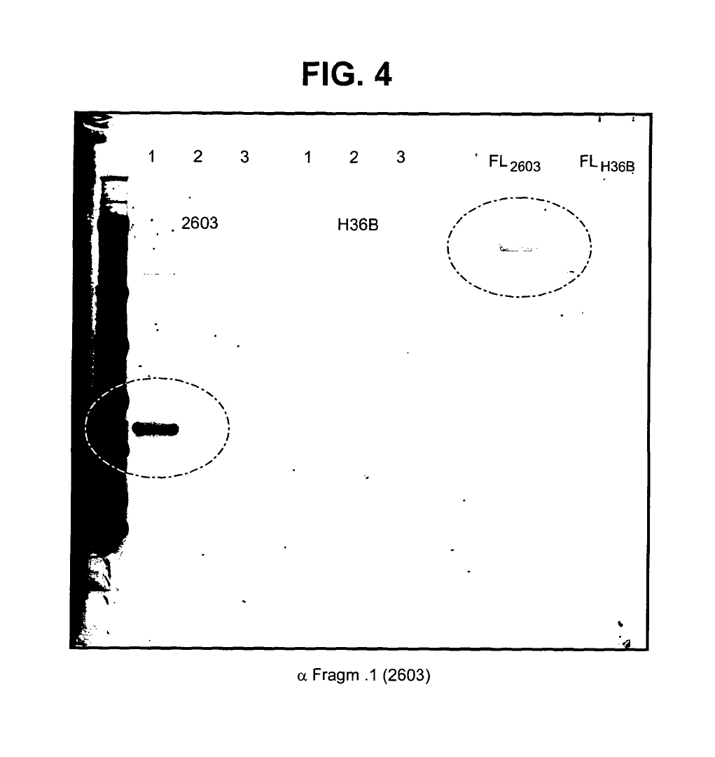

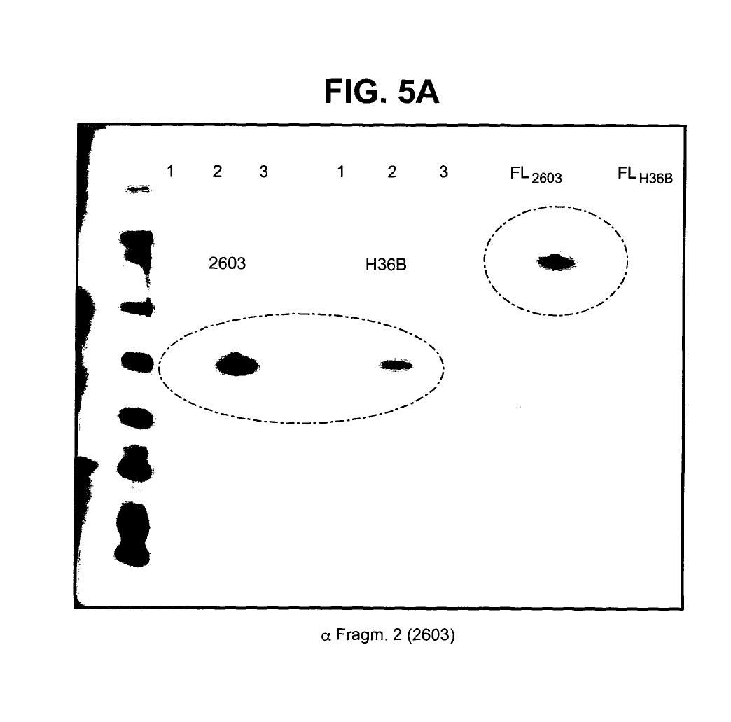

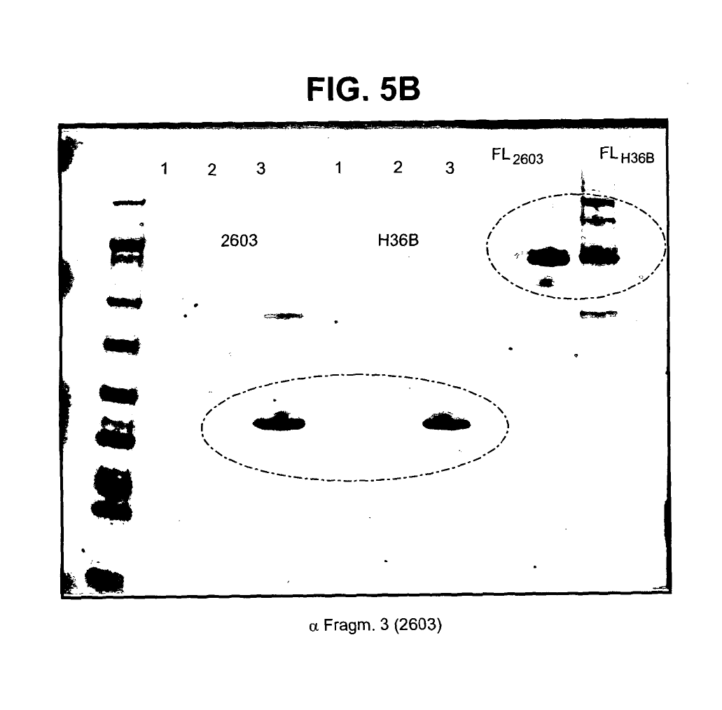

In a subsequent Western Blot analysis, antibodies raised against fragment 1 of the 2603 GBS67 variant only recognized recombinant fragment 1 from 2603 GBS67 and full-length GBS67 from 2603 (FIG. 4). These antibodies did not recognize fragment 1 from the H36B GBS67 variant or full-length GBS67 from H36B. In contrast, antibodies raised against fragment 2 from 2603 GBS67 recognized fragment 2 from H36B GBS67 and full-length H36B GBS67 (FIG. 5A) and antibodies raised against fragment 3 from 2603 GBS67 recognized fragment 3 from H36B GBS67 and full-length H36 GBS67 (FIG. 5B).

FACs analysis demonstrates that both fragments 2 and 3 of GBS67 are highly exposed on the surface of the GBS bacterium (see Table 4 below).

TABLE 4
Surface exposure of fragments 2 and 3

| | | FACS | | FACS |
|---|---|---|---|---|
| Group | Strain/serotype | Exposure | Strain/serotype | exposure |
| Fragment 1 2603 | 515 (Ia) | − | 5401 (II) | − |
| Fragment 2 2603 | 515 (Ia) | ++ | 5401 (II) | +++ |
| Fragment 3 2603 | 515 (Ia) | ++ | 5401 (II) | +++ |
| GBS67 2603 | 515 (Ia) | ++ | 5401 (II) | +++ |
| GBS67 H36B | 515 (Ia) | ++ | 5401 (II) | +++ |

The ability of fragments 1, 2 and 3 to induce cross-protection was then tested in vivo in a maternal immunization model. Female mice were immunized with fragment 1, 2 or 3 from GBS67 2603, with full-length GBS67 from 2603 or H36B, or with PBS. Pups were then challenged with the 5401 GBS strain expressing the GBS67 H36B variant. The results are shown in Table 5 below.

TABLE 5
Maternal immunization model results

| Group | Antigen | mcg/dose | Dead/treated | % survival |
|---|---|---|---|---|
| 1 | Fragment 1 (2603) | 20 | 54/60 | 10 |
| 2 | Fragment 2 (2603) | 20 | 37/60 | 38 |
| 3 | Fragment 3 (2603) | 20 | 22/45 | 51 |
| 4 | GBS67 (2603) | 20 | 25/54 | 54 |
| 5 | GBS67 (H36B) | 20 | 21/46 | 54 |
| 6 | PBS | 0 | 53/56 | 5 |

These results show that fragment 3 of the GBS67 2603 variant is able to confer the same cross-protection against 5401 GBS strain expressing the GBS67 H36B variant as full-length GBS67 2603 or full-length GBS67 H36B.

The ability of fragments 1, 2 and 3 of GBS67 2603 to induce protection against challenge with the 515 GBS strain expressing the GBS 67 2603 variant was confirmed by repeating the experiment described above except that pups were challenged with the 515 GBS strain, instead of the 5401 GBS strain. The results are shown in Table 6 below:

TABLE 6
Maternal immunization model results

| Group | Antigen | Dead/treated | % survival |
|---|---|---|---|
| 1 | Fragment 1 (2603) | 34/39 | 12 |
| 2 | Fragment 2 (2603) | 31/64 | 52 |
| 3 | Fragment 3 (2603) | 28/40 | 30 |
| 4 | GBS67 (2603) | 16/58 | 72 |
| 6 | PBS | 51/57 | 10 |

In a further experiment, female mice were immunized with fragment 1, 2 or 3 from GBS67 H36B, with full-length GBS67 from H36B, or with PBS. Pups were challenged with the 5401 GBS strain expressing the GBS67 H36B variant. The results are shown in Table 7 below.

TABLE 7
Maternal immunization model results

| Group | Antigen | Dead/treated | % survival |
|---|---|---|---|
| 1 | Fragment 1 (H36B) | 28/40 | 30 |
| 2 | Fragment 2 (H36B) | 16/50 | 68 |
| 3 | Fragment 3 (H36B) | 24/70 | 66 |
| 4 | GBS67 (H36B) | 21/48 | 56 |
| 5 | PBS | 43/69 | 38 |

Fragments 2 and 3 of GBS67 2603 and epitopes within these fragments may therefore be used in immunogenic compositions instead of full-length GBS67 2603 or full-length GBS67 H36B. Similarly, fragments 2 and 3 of GBS67 H36B and epitopes within these fragments may be used in immunogenic compositions instead of full-length GBS67 2603 or full-length GBS67 H36B.

### Materials and Methods

### Bioinformatics

The complete genome sequences of Streptococcus agalactiae strains 2603 V/R (V) and H36B (Ib) are available under Accession Numbers AE009948 and AAJS00000000. Pairwise sequence alignment was obtained by ClustalW algorithm.

In order to identify the putative architecture, we used Pfam program connected to NCBI-BLAST database. Secondary structure prediction was performed using PsiPred (Protein Structure Prediction Server; UCL Bioinformatic Group) software.

### Bacterial Strains and Growth Conditions

The GBS strains used in this work were 2603 V/R (serotype V), 515 (Ia), H36B (serotype Ib) and 5401 (II). Bacteria were grown at 37° C. in Todd Hewitt Broth (THB; Difco Laboratories) or in trypticase soy agar supplemented with 5% sheep blood.

### Cloning, Expression, Purification of Recombinant Proteins and Antisera

GBS strains 2603 and H36B were used as source of DNA for cloning the sequences coding for the single fragments (fragments 1, 2 and 3) of GBS67 2603 and H36B allelic variants. Genomic DNA was isolated by a standard protocol for gram-positive bacteria using a NucleoSpin Tissue kit (Macherey-Nagel) according to the manufacturer's instructions. Genes corresponding to each domain were cloned in the SpeedET or pET15-TEV vectors (N-terminal 6×HIS tag) by PIPE cloning method in E. coli HK100 strain (246). The oligos used are listed in Table 6. The resulting construct in pET15-TEV was checked for sequencing and then transformed into E. coli BL21(DE3) (Novagen). For the recombinant protein expression, the cultures were maintained at 25° C. for 5 h after induction with 1 mM IPTG for the pET clone or with 0.2% arabinose for the SpeedET clones. All recombinant proteins were purified by affinity chromatography. Briefly, cells were harvested by centrifugation and lysed in “lysis buffer”, containing 10 mM imidazole, 1 mg\ml lysozyme, 0.5 mg\ml DNAse and COMPLETE inhibitors cocktail (Roche) in PBS. The lysate was clarified by centrifugation and applied onto His-Trap HP column (Armesham Biosciences) pre-equilibrated in PBS containing 10 mM imidazole. Protein elution was performed using the same buffer containing 250 mM imidazole, after two wash steps using 20 mM and 50 mM imidazole buffers. Protein concentration of the pure fractions was estimated using BCA assay (PIERCE).

Antisera specific for each protein were produced by immunizing CD1 mice with the purified recombinant proteins as previously described [247]. Protein-specific immune responses (total Ig) in pooled sera were monitored by ELISA.

The full length recombinant GBS67 proteins, corresponding to 2603 and H36B allelic variants (TIGR annotation SAG_{—}1408 and SAI_{—}1512, respectively), were produced as previously reported [2, 247].

### Immunoblotting

10 ng of each purified protein were separated by 4-12% NuPage Novex pre-cast gels (Invitrogen) and electroblotted onto nitrocellulose membranes using the iBlot™ Dry Blotting System (Invitrogen). After blocking in 1× phosphate-buffered saline (PBS: 140 mM NaCl, 2.7 mM KCl, 10 mM Na2HPO4 and 1.8 mm KH2PO4, pH 7.3) containing 0.05% Tween 20 and 10% skim milk for 1 h at room temperature, membranes were incubated for 1 h at room temperature (RT) with primary antibodies diluted 1:500. After washing three times in PBS containing 0.05% Tween 20 (PBST), the membranes were incubated for 1 h with horseradish peroxidase-conjugated secondary antibodies (Dako). Positive bands were visualized with the Opti-4CN Substrate Kit (Bio-Rad).

### ELISA

Antigen-specific antibody responses were detected by ELISA, by using of 100 ng of purified recombinant antigens per well. IgG antibody titers were calculated by comparing the response curve of test serum samples with that of reference serum samples by using a reference line calculation program. The reference serum samples were a pool of serum samples obtained from mice immunized with the purified recombinant antigen, to which an arbitrary titer of 150,000 EU/mL was assigned.

### FACS

Mouse sera raised against purified recombinant proteins were analyzed on whole bacteria by flow cytometry to evaluate the surface-exposure of the single domains. Exponential phase bacterial cells were fixed in the presence of 0.08% (wt/vol) paraformaldehyde and incubated for 1 h at 37° C. Fixed bacteria were then washed once with PBS, resuspended in Newborn Calf Serum (Sigma) and incubated for 20 min. at 25° C. The cells were then incubated for 1 hour at 4° C. in pre-immune or immune sera, diluted 1:200 in dilution buffer (PBS, 20% Newborn Calf Serum, 0.1% BSA). Cells were washed in PBS-01% BSA and incubated for a further 1 h at 4° C. with a 1:100 dilution of R-Phycoerythrin conjugated F(ab)2 goat anti-mouse IgG (Jackson ImmunoResearch Laboratories; Inc.). After washing, cells were resuspended in PBS and analyzed with a FACS Calibur apparatus (Becton Dickinson, Franklin Lakes, N.J.) using FlowJo Software (Tree Star, Ashland, Oreg.). Data are expressed as the difference in fluorescence between cells stained with immune sera versus pre-immune sera.

### Mouse Active Maternal Immunization Model

A maternal immunization/neonatal pup challenge model of GBS infection was used to verify the protective efficacy of the produced proteins in mice, as previously described [247]. Briefly, CD-1 female mice (6-8 weeks old) were immunized on days 1 (in CFA), 21 and 35 (IFA) with either PBS or 20 mg of recombinant protein and were then bred 3 days after the last immunization. Within 48 h of birth, pups were injected intraperitoneally with a dose of different GBS strains calculated to cause 90% lethality.

Survival of pups was monitored for 2 days after challenge. Statistical analysis was performed using Fisher's exact test. All animal studies were performed according to guidelines of the Istituto Superiore di Sanità (Italy).

TABLE 6
Primers used to clone GBS67 fragments

| Primer | Sequence (5′to 3′) | gene amplified |
|---|---|---|
| Fr 1-2603 for | CTGTACTTCCAGGGCAATACCAATGTTTTAGGGGAA (SEQ ID NO: 68) | fragment coding for |
| Fr 1-2603 rev | AATTAAGTCGCGTTATTTTCCACTGACAGTTAACTC (SEQ ID NO: 69) | aa24-217 of GBS67- |
| | | 2603 variant |
| Fr 2-2603 for | CTGTACTTCCAGGGCACCATAGTAAAACCAGTGGAC (SEQ ID NO: 70) | fragment coding for |
| Fr 2-2603 rev | AATTAAGTCGCGTTATCCATTACCAAGCTGTAAATT (SEQ ID NO: 71) | aa218-615aa of |
| | | GBS67-2603 variant |
| Fr 3-2603 for | CTGTACTTCCAGGGCCAAACATTACAGCCAAGTGAT (SEQ ID NO: 72) | fragment coding for |
| Fr 3-2603 rev | AATTAAGTCGCGTTATCCTTTCCCACCTGTCATAGG (SEQ ID NO: 73) | aa616-866 of GBS67- |
| | | 2603 variant |
| Fr 1-H36B for | CTGTACTTCCAGGGCAATACCAATGTTTTAGGGGAA (SEQ ID NO: 74) | fragment coding for |
| Fr 1-H36B rev | AATTAAGTCGCGTTATTTACCGCTAACAGTTAACTC (SEQ ID NO: 75) | aa24-217 of GBS67- |
| | | H36B variant |
| Fr 2-H36B for | CTGTACTTCCAGGGCTCCATAATAAAAACTATAAATAAAG (SEQ ID NO: 76) | fragment coding for |
| Fr 2-H36B rev | AATTAAGTCGCGTTATCCGTTGCCAAGATGTAAATT (SEQ ID NO: 77) | aa218-610 of GBS67- |
| | | H36B variant |
| Fr 3-H36B for | CTGTACTTCCAGGGCCAAACATTGCAACCAAGTGAT (SEQ ID NO: 78) | fragment coding for |
| Fr 3-H36B rev | AATTAAGTCGCGTTATCCTTTCCCACCTGTCATC (SEQ ID NO: 79) | aa611-861aa of |
| | | GBS67-H36B variant |

### REFERENCES

[1] Rosini et al, Molecular Microbiology, 2006, 61(1): 126-141
[2] Margarit et al, Journal of Infectious Diseases, 2009, 199: 108-115
[3] Tettelin et al, Proc. Natl. Acad. Sci. U.S.A. 102 (39), 13950-13955 (2005)
[4] Needleman & Wunsch (1970) J. Mol. Biol. 48, 443-453.
[5] Rice et al. (2000) Trends Genet 16:276-277.
[6] Bodanszky (1993) Principles of Peptide Synthesis (ISBN: 0387564314).
[7] Fields et al. (1997) Meth Enzymol 289: Solid-Phase Peptide Synthesis. ISBN: 0121821900.
[8] Chan & White (2000) Fmoc Solid Phase Peptide Synthesis. ISBN: 0199637245.
[9] Kullmann (1987) Enzymatic Peptide Synthesis. ISBN: 0849368413.
[10] Ibba (1996) Biotechnol Genet Eng Rev 13:197-216.
[11] U.S. Pat. No. 5,707,829
[12] Vaccine Design (1995) eds. Powell & Newman. ISBN: 030644867X. Plenum.
[13] WO90/14837.
[14] WO90/14837.
[15] Podda & Del Giudice (2003) Expert Rev Vaccines 2:197-203.
[16] Podda (2001) Vaccine 19: 2673-2680.
[17] Vaccine Design: The Subunit and Adjuvant Approach (eds. Powell & Newman) Plenum Press 1995 (ISBN 0-306-44867-X).
[18] Vaccine Adjuvants: Preparation Methods and Research Protocols (Volume 42 of Methods in Molecular Medicine series). ISBN: 1-59259-083-7. Ed. O'Hagan.
[19] Allison & Byars (1992) Res Immunol 143:519-25.
[20] Hariharan et al. (1995) Cancer Res 55:3486-9.
[21] US-2007/014805.
[22] WO95/11700.
[23] U.S. Pat. No. 6,080,725.
[24] WO2006/113373.
[25] WO2005/097181.
[26] U.S. Pat. No. 5,057,540.
[27] WO96/33739.
[28] EP-A-0109942.
[29] WO96/11711.
[30] WO00/07621.
[31] Barr et al. (1998) Advanced Drug Delivery Reviews 32:247-271.
[32] Sjolanderet et al. (1998) Advanced Drug Delivery Reviews 32:321-338.
[33] Niikura et al. (2002) Virology 293:273-280.
[34] Lenz et al. (2001) J Immunol 166:5346-5355.
[35] Pinto et al. (2003) J Infect Dis 188:327-338.
[36] Gerber et al. (2001) J Virol 75:4752-4760.
[37] WO03/024480.
[38] WO03/024481.
[39] Gluck et al. (2002) Vaccine 20:B10-B16.
[40] EP-A-0689454.
[41] Johnson et al. (1999) Bioorg Med Chem Lett 9:2273-2278.
[42] Evans et al. (2003) Expert Rev Vaccines 2:219-229.
[43] Meraldi et al. (2003) Vaccine 21:2485-2491.
[44] Pajak et al. (2003) Vaccine 21:836-842.
[45] Kandimalla et al. (2003) Nucleic Acids Research 31:2393-2400.
[46] WO02/26757.
[47] WO99/62923.
[48] Krieg (2003) Nature Medicine 9:831-835.
[49] McCluskie et al. (2002) FEMS Immunology and Medical Microbiology 32:179-185.
[50] WO98/40100.
[51] U.S. Pat. No. 6,207,646.
[52] U.S. Pat. No. 6,239,116.
[53] U.S. Pat. No. 6,429,199.
[54] Kandimalla et al. (2003) Biochemical Society Transactions 31 (part 3):654-658.
[55] Blackwell et al. (2003) J Immunol 170:4061-4068.
[56] Krieg (2002) Trends Immunol 23:64-65.
[57] WO01/95935.
[58] Kandimalla et al. (2003) BBRC 306:948-953.
[59] Bhagat et al. (2003) BBRC 300:853-861.
[60] WO03/035836.
[61] Schellack et al. (2006) Vaccine 24:5461-72.
[62] WO95/17211.
[63] WO98/42375.
[64] Beignon et al. (2002) Infect Immun 70:3012-3019.
[65] Pizza et al. (2001) Vaccine 19:2534-2541.
[66] Pizza et al. (2000) Int J Med Microbiol 290:455-461.
[67] Scharton-Kersten et al. (2000) Infect Immun 68:5306-5313.
[68] Ryan et al. (1999) Infect Immun 67:6270-6280.
[69] Partidos et al. (1999) Immunol Lett 67:209-216.
[70] Peppoloni et al. (2003) Expert Rev Vaccines 2:285-293.
[71] Pine et al. (2002) J Control Release 85:263-270.
[72] Tebbey et al. (2000) Vaccine 18:2723-34.
[73] Domenighini et al. (1995) Mol Microbiol 15:1165-1167.
[74] WO99/40936.
[75] WO99/44636.
[76] Singh et al] (2001) J Cont Release 70:267-276.
[77] WO99/27960.
[78] U.S. Pat. No. 6,090,406.
[79] U.S. Pat. No. 5,916,588.
[80] EP-A-0626169.
[81] WO99/52549.
[82] WO01/21207.
[83] WO01/21152.
[84] Andrianov et al. (1998) Biomaterials 19:109-115.
[85] Payne et al. (1998) Adv Drug Delivery Review 31:185-196.
[86] Stanley (2002) Clin Exp Dermatol 27:571-577.
[87] Jones (2003) Curr Opin Investig Drugs 4:214-218.
[88] WO99/11241.
[89] WO94/00153.
[90] WO98/57659.
[91] European patent applications 0835318, 0735898 and 0761231.
[92] Donnelly et al. (1997) Annu Rev Immunol 15:617-648.
[93] Strugnell et al. (1997) Immunol Cell Biol 75(4):364-369.
[94] Cui (2005) Adv Genet 54:257-89.
[95] Robinson & Torres (1997) Seminars in Immunol 9:271-283.
[96] Brunham et al. (2000) J Infect Dis 181 Suppl 3:S538-43.
[97] Svanholm et al. (2000) Scand J Immunol 51(4):345-53.
[98] DNA Vaccination—Genetic Vaccination (1998) eds. Koprowski et al. (ISBN 3540633928).
[99] Gene Vaccination: Theory and Practice (1998) ed. Raz (ISBN 3540644288).
[100] Findeis et al., Trends Biotechnol. (1993) 11:202
[101] Chiou et al. (1994) Gene Therapeutics: Methods And Applications Of Direct Gene Transfer. ed. Wolff
[102] Wu et al., J. Biol. Chem. (1988) 263:621
[103] Wu et al., J. Biol. Chem. (1994) 269:542
[104] Zenke et al., Proc. Natl. Acad. Sci. (USA) (1990) 87:3655
[105] Wu et al., J. Biol. Chem. (1991) 266:338
[106] Jolly, Cancer Gene Therapy (1994) 1:51
[107] Kimura, Human Gene Therapy (1994) 5:845
[108] Connelly, Human Gene Therapy (1995) 1:185
[109] Kaplitt, Nature Genetics (1994) 6:148
[110] WO 90/07936.
[111] WO 94/03622.
[112] WO 93/25698.
[113] WO 93/25234.
[114] U.S. Pat. No. 5,219,740.
[115] WO 93/11230.
[116] WO 93/10218.
[117] U.S. Pat. No. 4,777,127.
[118] GB Patent No. 2,200,651.
[119] EP-A-0345242.
[120] WO 91/02805.
[121] WO 94/12649.
[122] WO 93/03769.
[123] WO 93/19191.
[124] WO 94/28938.
[125] WO 95/11984.
[126] WO 95/00655.
[127] Curiel, Hum. Gene Ther. (1992) 3:147
[128] Wu, J. Biol. Chem. (1989) 264:16985
[129] U.S. Pat. No. 5,814,482.
[130] WO 95/07994.
[131] WO 96/17072.
[132] WO 95/30763.
[133] WO 97/42338.
[134] WO 90/11092.
[135] U.S. Pat. No. 5,580,859
[136] U.S. Pat. No. 5,422,120
[137] WO 95/13796.
[138] WO 94/23697.
[139] WO 91/14445.
[140] EP-0524968.
[141] Philip, Mol. Cell Biol. (1994) 14:2411
[142] Woffendin, Proc. Natl. Acad. Sci. (1994) 91:11581
[143] U.S. Pat. No. 5,206,152.
[144] WO 92/11033.
[145] U.S. Pat. No. 5,149,655.
[146] Zwijnenburg et al. (2001) J Infect Dis 183:1143-6.
[147] Tettelin et al, Proc. Natl. Acad. Sci. U.S.A. 99 (19), 12391-12396 (2002)
[148] WO09/101,403
[149] WO2006/130328
[150] Lewis et al. (2004) PNAS USA 101:11123-8.
[151] WO2006/050341.
[152] Paoletti et al. (1992) J Clin Invest 89:203-9
[153] WO96/40795
[154] Wessels et al. (1989) Infect Immun 57:1089-94.
[155] WO2006/082527.
[156] Paoletti et al. (1990) J Biol Chem 265:18278-83.
[157] Wessels et al. (1990) J Clin Invest 86:1428-33.
[158] Paoletti et al. (1992) Infect Immun 60:4009-14.
[159] Wessels et al. (1987) Proc Natl Acad Sci USA 84:9170-4.
[160] Wang et al. (2003) Vaccine 21:1112-7.
[161] Wessels et al. (1993) Infect Immun 61:4760-6
[162] Wessels et al. (1995) J Infect Dis 171:879-84.
[163] Baker et al. (2004) J Infect Dis 189:1103-12.
[164] U.S. Pat. No. 4,356,170.
[165] Paoletti & Kasper (2003) Expert Opin Biol Ther 3:975-84.
[166] WO2005/000346
[167] Anonymous (January 2002) Research Disclosure, 453077.
[168] Anderson (1983) Infect Immun 39(1):233-238.
[169] Anderson et al. (1985) J Clin Invest 76(1):52-59.
[170] EP-A-0372501.
[171] EP-A-0378881.
[172] EP-A-0427347.
[173] WO93/17712.
[174] WO94/03208.
[175] WO98/58668.
[176] EP-A-0471177.
[177] WO91/01146.
[178] Falugi et al. (2001) Eur J Immunol 31:3816-3824.
[179] Baraldo et al. (2004) Infect Immun 72(8):4884-7.
[180] EP-A-0594610.
[181] Ruan et al. (1990) J Immunol 145:3379-3384.
[182] WO00/56360.
[183] WO01/72337.
[184] WO00/61761.
[185] WO00/33882
[186] WO99/42130.
[187] U.S. Pat. No. 4,761,283.
[188] U.S. Pat. No. 4,356,170.
[189] U.S. Pat. No. 4,882,317.
[190] U.S. Pat. No. 4,695,624.
[191] Mol. Immunol., 1985, 22, 907-919
[192] EP-A-0208375.
[193] Bethell G. S. et al., J. Biol. Chem., 1979, 254, 2572-4
[194] Hearn M. T. W., J. Chromatogr., 1981, 218, 509-18
[195] WO00/10599.
[196] Gever et al., Med. Microbiol. Immunol, 165: 171-288 (1979).
[197] U.S. Pat. No. 4,057,685.
[198] U.S. Pat. Nos. 4,673,574; 4,761,283; 4,808,700.
[199] U.S. Pat. No. 4,459,286.
[200] U.S. Pat. No. 4,965,338.
[201] U.S. Pat. No. 4,663,160.
[202] WO2007/000343.
[203] Vaccines. (eds. Plotkin & Orenstein). 4th edition, 2004, ISBN: 0-7216-9688-0.
[204] Rappuoli et al. (1991) TIBTECH 9:232-238.
[205] Harper et al. (2004) Lancet 364(9447):1757-65.
[206] Brandt et al. (2006) J Antimicrob Chemother. 58(6):1291-4. Epub 2006 Oct. 26
[207] Winter et al., (1991) Nature 349:293-99
[208] U.S. Pat. No. 4,816,567.
[209] Inbar et al., (1972) Proc. Natl. Acad. Sci. U.S.A. 69:2659-62.
[210] Ehrlich et al., (1980) Biochem 19:4091-96.
[211] Huston et al., (1988) Proc. Natl. Acad. Sci. U.S.A. 85:5897-83.
[212] Pack et al., (1992) Biochem 31, 1579-84.
[213] Cumber et al., (1992) J. Immunology 149B, 120-26.
[214] Riechmann et al., (1988) Nature 332, 323-27.
[215] Verhoeyan et al., (1988) Science 239, 1534-36.
[216] GB 2,276,169.
[217] Gennaro (2000) Remington: The Science and Practice of Pharmacy. 20th edition, ISBN: 0683306472.
[218] Methods In Enzymology (S. Colowick and N. Kaplan, eds., Academic Press, Inc.)
[219] Handbook of Experimental Immunology, Vols. I-IV (D. M. Weir and C. C. Blackwell, eds, 1986, Blackwell Scientific Publications)
[220] Sambrook et al. (2001) Molecular Cloning: A Laboratory Manual, 3rd edition (Cold Spring Harbor Laboratory Press).
[221] Handbook of Surface and Colloidal Chemistry (Birdi, K. S. ed., CRC Press, 1997)
[222] Ausubel et al. (eds) (2002) Short protocols in molecular biology, 5th edition (Current Protocols).
[223] Molecular Biology Techniques: An Intensive Laboratory Course, (Ream et al., eds., 1998, Academic Press)
[224] PCR (Introduction to Biotechniques Series), 2nd ed. (Newton & Graham eds., 1997, Springer Verlag)
[225] Geysen et al. (1984) PNAS USA 81:3998-4002.
[226] Carter (1994) Methods Mol Biol 36:207-23.
[227] Jameson, B A et al. 1988, CABIOS 4(1):181-186.
[228] Raddrizzani & Hammer (2000) Brief Bioinform 1(2):179-89.
[229] Bublil et al. (2007) Proteins 68(1):294-304.
[230] De Lalla et al. (1999) J. Immunol. 163:1725-29.
[231] Kwok et al. (2001) Trends Immunol 22:583-88.
[232] Brusic et al. (1998) Bioinformatics 14(2):121-30
[233] Meister et al. (1995) Vaccine 13(6):581-91.
[234] Roberts et al. (1996) AIDS Res Hum Retroviruses 12(7):593-610.
[235] Maksyutov & Zagrebelnaya (1993) Comput Appl Biosci 9(3):291-7.
[236] Feller & de la Cruz (1991) Nature 349(6311):720-1.
[237] Hopp (1993) Peptide Research 6:183-190.
[238] Welling et al. (1985) FEBS Lett. 188:215-218.
[239] Davenport et al. (1995) Immunogenetics 42:392-297.
[240] Tsurui & Takahashi (2007) J Pharmacol Sci. 105(4):299-316.
[241] Tong et al. (2007) Brief Bioinform. 8(2):96-108.
[242] Schirle et al. (2001) J Immunol Methods. 257(1-2):1-16.
[243] Chen et al. (2007) Amino Acids 33(3):423-8.
[244] Current Protocols in Molecular Biology (F. M. Ausubel et al., eds., 1987) Supplement 30
[245] Smith & Waterman (1981) Adv. Appl. Math. 2: 482-489.
[246] Klock et al, (2008), Proteins, 71:982-994
[247] Maione et al (2005), Science, 309: 148-150

1. A polypeptide consisting of:
i) a fragment of at least 7 contiguous amino acids from SEQ ID NO:1, wherein said fragment comprises an epitope from the amino acid sequence of SEQ ID NO:4;
ii) a fragment of at least 7 contiguous amino acids from an amino acid sequence having at least 90% identity to SEQ ID NO:1, wherein said fragment comprises an epitope having at least 90% identity to an epitope from the amino acid sequence of SEQ ID NO:4;
iii) a fragment of at least 7 contiguous amino acids from SEQ ID NO:5, wherein said fragment comprises an epitope from the amino acid sequence of SEQ ID NO:8; or
iv) a fragment of at least 7 contiguous amino acids from an amino acid sequence having at least 90% identity to SEQ ID NO:5, wherein said fragment comprises an epitope having at least 90% identity to an epitope from the amino acid sequence of SEQ ID NO:8.

2. A polypeptide according to claim 1 that consists of:
i) a fragment from SEQ ID NO:1, wherein said fragment comprises the amino acid sequence of SEQ ID NO:4;
ii) a fragment from an amino acid sequence having 90% identity to SEQ ID NO:1, wherein said fragment comprises an amino acid sequence having at least 90% identity to SEQ ID NO:4;
iii) a fragment from SEQ ID NO:5, wherein said fragment comprises the amino acid sequence of SEQ ID NO:8; or
iv) a fragment from an amino acid sequence having at least 90% identity to SEQ ID NO:5, wherein said fragment comprises an amino acid sequence having at least 90% identity to SEQ ID NO:8.

3. A polypeptide according to claim 1 that comprises:
i) a fragment of SEQ ID NO:1 comprising the amino acid sequence of SEQ ID NO:4; or
ii) a fragment from SEQ ID NO:5 comprising the amino acid sequence of SEQ ID NO:8.

4. A polypeptide comprising an amino acid sequence:
A-{-X-L}ₙ-B
wherein:
each X is a polypeptide consisting of:
i) a fragment of at least 7 contiguous amino acids from SEQ ID NO:1, wherein said fragment comprises an epitope from the amino acid sequence of SEQ ID NO:4;
ii) a fragment of at least 7 contiguous amino acids from an amino acid sequence having at least 90% identity to SEQ ID NO:1, wherein said fragment comprises an epitope having at least 90% identity to an epitope from the amino acid sequence of SEQ ID NO:4;
iii) a fragment of at least 7 contiguous amino acids from SEQ ID NO:5, wherein said fragment comprises an epitope from the amino acid sequence of SEQ ID NO:8; or
iv) a fragment of at least 7 contiguous amino acids from an amino acid sequence having at least 90% identity to SEQ ID NO:5, wherein said fragment comprises an epitope having at least 90% identity to an epitope from the amino acid sequence of SEQ ID NO:8;
L is an optional linker amino acid sequence;
A is an optional N terminal amino acid sequence;
B is an optional C terminal amino acid sequence; and
n is an integer of 1 or more.

5. A polypeptide according to claim 1 that elicits an antibody response comprising antibodies that bind to the wild-type GBS protein having amino acid sequence of SEQ ID NO:1 and to the amino acid sequence of the wild-type GBS protein having the amino acid sequence of SEQ ID NO:5.

6. A polypeptide according to claim 1 that elicits an antibody response comprising antibodies that bind to the wild-type GBS protein having amino acid sequence SEQ ID NO: 9 (strain CJB111), the wild-type GBS protein having amino acid sequence SEQ ID NO: 13 (strain 515), the wild-type GBS protein having amino acid sequence SEQ ID NO: 17 (strain NEM316), the wild-type GBS protein having amino acid sequence SEQ ID NO: 21 (strain DK21), and the wild-type GBS protein having amino acid sequence SEQ ID NO: 25 (strain CJB110).

7. A conjugate comprising a saccharide moiety and a carrier protein moiety, wherein the carrier protein moiety comprises a polypeptide according to claim 1.

8. A nucleic acid encoding a polypeptide according to claim 1.

9. An immunogenic composition comprising:
(a) a first polypeptide consisting of:
i) a fragment of at least 7 contiguous amino acids from SEQ ID NO:1, wherein said fragment comprises an epitope from the amino acid sequence of SEQ ID NO:4;
ii) a fragment of at least 7 contiguous amino acids from an amino acid sequence having at least 90% identity to SEQ ID NO:1, wherein said fragment comprises an epitope having at least 90% identity to an epitope from the amino acid sequence of SEQ ID NO:4;
iii) a fragment of at least 7 contiguous amino acids from SEQ ID NO:5, wherein said fragment comprises an epitope from the amino acid sequence of SEQ ID NO:8; or
iv) a fragment of at least 7 contiguous amino acids from an amino acid sequence having at least 90% identity to SEQ ID NO:5, wherein said fragment comprises an epitope having at least 90% identity to an epitope from the amino acid sequence of SEQ ID NO:8, a conjugate comprising a saccharide moiety and a carrier protein moiety,
wherein the carrier protein moiety comprises the first polypeptide;
(b) a second polypeptide comprising an amino acid sequence:
A-{-X-L}ₙ-B
wherein:
X is the first polypeptide
L is an optional linker amino acid sequence;
A is an optional N terminal amino acid sequence;
B is an optional C terminal amino acid sequence; and
n is an integer of 1 or more;
(c) a second conjugate comprising a saccharide moiety and a carrier protein moiety, wherein the carrier protein moiety comprises the second polypeptide;
(d) a nucleic acid encoding the first polypeptide; or
(e) a nucleic acid encoding the second polypeptide.

10-11. (canceled)

12. A method of treating an infection caused by GBS in a mammal comprising administering to the mammal an effective amount of the immunogenic composition of claim 9.

13. A bacterium which expresses the polypeptide of claim 1.

14. The method of claim 12, wherein the infection is meningitis.
